(19) United States
(12) Patent Application Publication
(10) Pub. No.: US 2021/0015959 A1
(43) Pub. Date: Jan. 21, 2021

(54) ULTRAVIOLET IRRADIATION DEVICE, ULTRAVIOLET IRRADIATION METHOD, ILLUMINATION DEVICE, AND ULTRAVIOLET IRRADIATION SYSTEM

(71) Applicant: SUN ENERGY CORPORATION, Minoh-shi, Osaka (JP)

(72) Inventors: Narihide GOSEKI, Kasukabe-shi (JP); Hirokazu UMEKAGE, Minoh-shi (JP); Atsushi NAGAO, Minoh-shi (JP); Takashi KAWABATA, Hasuda-shi (JP)

(21) Appl. No.: 17/042,850

(22) PCT Filed: Mar. 29, 2018
(86) PCT No.: PCT/JP2018/013195

§ 371 Date: Sep. 28, 2020

(51) Int. Cl. A61L 9/20; A61B 90/30; A61B 90/40

(52) U.S. Cl. CPC A61L 9/20; A61B 90/30; A61L 2209/212; A61L 2209/12; A61B 90/40

(57) ABSTRACT

Provided are an ultraviolet irradiation device, an ultraviolet irradiation method, an illumination device, and an ultraviolet irradiation system capable of efficiently and safely disinfecting an area to be disinfected and capable of preventing reduction in the operating rate of (equipment, etc., in) the area to be disinfected by maintaining the disinfected state. The ultraviolet irradiation device includes an ultraviolet irradiation unit capable of outputting ultraviolet having a predetermined dominant wavelength, and a drive control unit. The drive control unit performs time control of ultraviolet irradiation and non-irradiation by the ultraviolet irradiation unit on the basis of time necessary to disinfect the area S to be disinfected before or during operation and bacterial growth time after the disinfection.

### CROSS REFERENCE TO RELATED APPLICATIONS

This application is a U.S. National Phase application of International Application No. PCT/JP2018/013195, filed on Mar. 29, 2018. The entire disclosure of the above application is expressly incorporated by reference herein.

### BACKGROUND

### Technical Field

The present invention relates to an ultraviolet irradiation device, an ultraviolet irradiation method, an illumination device, and an ultraviolet irradiation system.

### Related Art

In a work area (working facility) in which the number of bacteria is controlled, such as an operating room, an aseptic room, or a clean room, disinfection treatment via, for example, formalin fumigation, ethylene oxide gas (EOG) sterilization, or cleaning with a disinfectant is performed before and after a work in conventional techniques. In this manner, cleanliness is normally being ensured.

For an electronic device installed in a work area in which the number of bacteria is controlled (for example, a shadowless lamp in an operating room), there is a possibility that the inside of the electronic device is contaminated by air flowing into the electronic device. Disinfection treatment such as cleanup or sanitization, however, cannot be performed directly by opening the electronic device. Thus, a device for disinfecting the inside of a case by providing an ultraviolet LED in the case of the electronic device and irradiating the inside of the case with ultraviolet light has been known conventionally (see Japanese Patent Application Laid-Open No. 2007-44334, for example).

Such disinfection treatment via, for example, formalin fumigation or EOG sterilization, however, is extensive, costly, and harmful to the human body. Thus, such disinfection treatment needs to be performed during time other than time for performing an originally intended work (during the operation of the work area). This is a major factor for reducing the operating rate of the work area and/or equipment in the work area, for example.

Since cleaning with a disinfectant is manually done by a worker, such work takes time, thus resulting in similar problems such as reduction in the operating rate of equipment in the work area, and increase in labor cost.

Taking an operating room as an example of the work area, bacteria are introduced into the operating room as a result of doctors, nurses, patients, etc., entering and leaving the operating room even after the disinfection treatment is performed. Thus, to maintain the state immediately after the disinfection treatment up to the point of surgery during which it is essentially desired to have cleanliness reliably (a substantially sterilized state) is substantially impossible.

The technique described in Japanese Patent Application Laid-Open No. 2007-44334, for example, is for locally disinfecting the inside of an electronic device (such as a shadowless lamp) used in an operating room, for example. Such a technique cannot disinfect the work area to be disinfected (such as the ceiling, the floor, the walls, and the space).

Under such a work environment having a possibility that bacteria having effects on the human body grow, infection prevention is essential. While there is a need for disinfecting the whole work area safely, improving the operating rate of equipment, etc., and reducing a maintenance management cost are being demanded strongly. Performing infection control and cleanliness management in a rational way is strongly needed.

In view of the foregoing circumstances, it is an object of the present invention to provide an ultraviolet irradiation device, an ultraviolet irradiation method, an illumination device, and an ultraviolet irradiation system capable of efficiently and safely disinfecting a work area to be disinfected (an area to be disinfected) and capable of preventing reduction in the operating rate of (equipment, etc., in) the area to be disinfected by maintaining the disinfected state.

### SUMMARY

An aspect of the present invention provides an ultraviolet irradiation device configured to perform disinfection by irradiating an area to be disinfected with ultraviolet, including: an ultraviolet irradiation unit capable of outputting ultraviolet having a predetermined dominant wavelength; and a drive control unit. The drive control unit performs time control of ultraviolet irradiation and non-irradiation by the ultraviolet irradiation unit on the basis of time necessary to disinfect the area to be disinfected before or during operation and bacterial growth time after the disinfection.

Another aspect of the present invention provides an ultraviolet irradiation method for performing disinfection by irradiating an area to be disinfected with ultraviolet, including performing time control of irradiation and non-irradiation with ultraviolet having a predetermined dominant wavelength on the basis of time necessary to disinfect the area to be disinfected during operation and bacterial growth time after the disinfection.

Still another aspect of the present invention provides an illumination device including the above-described ultraviolet irradiation device and an illumination light source.

Still another aspect of the present invention provides an ultraviolet illumination system including: the above-described ultraviolet irradiation device; and a management unit configured to manage entry and exit of a person to and from the area to be disinfected. The drive control unit performs control of the ultraviolet irradiation device in conjunction with entry and exit management made by the management unit.

### Advantageous Effects of Invention

The present invention can yield excellent effects such that the ultraviolet irradiation device, the ultraviolet irradiation method, the illumination device, and the ultraviolet irradiation system capable of efficiently and safely disinfecting the area to be disinfected and capable of preventing reduction in the operating rate of (equipment, etc., in) the area to be disinfected by maintaining the disinfected state can be provided.

### BRIEF DESCRIPTION OF THE DRAWINGS

FIGS. 1A-1C show diagrams for explaining an ultraviolet irradiation device according to an embodiment of the present invention: FIG. 1A is a schematic view; FIG. 1B is a sectional side view; and FIG. 1C is a timing chart showing turning-on and turning-off control.

FIG. 2 is a flow diagram for explaining an ultraviolet irradiation method according to the embodiment of the present invention.

FIG. 3A is a graph showing a relationship between an output wavelength distribution of a UV lamp included in the ultraviolet irradiation device according to the embodiment of the present invention and a UV absorption rate of DNA, and

FIG. 3B is a graph showing a relationship between a UV absorption rate of DNA and a bactericidal rate by UV.

FIG. 4 is a table showing a list of energy amounts necessary for inactivation by UV according to kinds of bacteria.

FIG. 5 shows graphs each representing a relationship between a bactericidal rate and a UV irradiation amount for a kind of bacteria.

FIG. 6 is a graph showing a relationship between an irradiation distance of the UV lamp included in the ultraviolet irradiation device according to the embodiment of the present invention and UV illuminance.

FIG. 7A is a graph showing change in doubling time associated with change in incubation temperature for Bacillus subtilis and Clostridium perfringens, and FIG. 7B is a table showing doubling time and average cell lengths associated with change in incubation temperature for Bacillus subtilis and Clostridium perfringens.

FIGS. 8A and 8B show schematic views each illustrating an example of the layout and turning-on and turning-off control of the ultraviolet irradiation device according to the embodiment of the present invention.

FIG. 9 is an external perspective view of an illumination device according to the embodiment of the present invention.

FIG. 10 is an external perspective view of the illumination device according to the embodiment of the present invention.

FIG. 11 is a sectional side view of the illumination device according to the embodiment of the present invention.

FIG. 12 is a schematic view showing ultraviolet irradiation directions of the illumination device according to the embodiment of the present invention.

FIG. 13 is a schematic circuit diagram of an illumination device according to an embodiment of the present invention.

### DETAILED DESCRIPTION

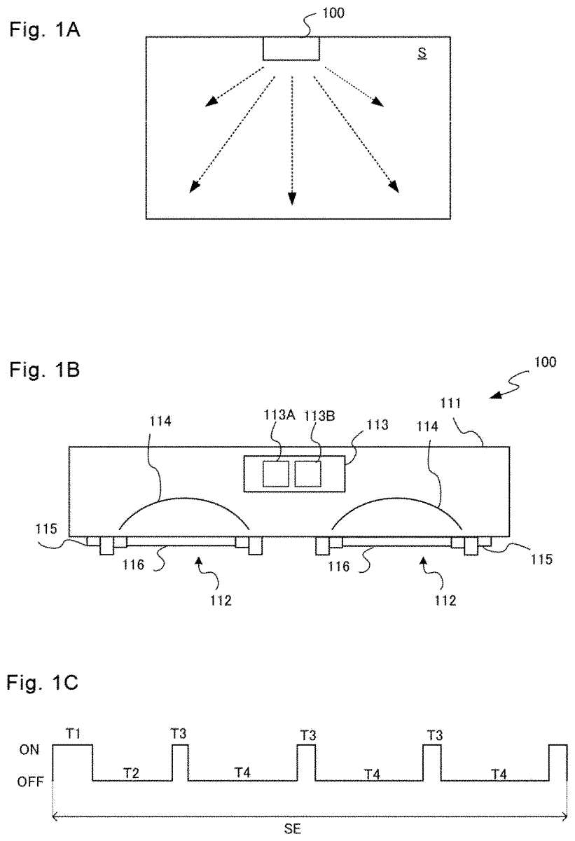

An embodiment of the present invention will be described below with reference to FIGS. 1 to 13.

### Ultraviolet Irradiation Device

FIGS. 1A-1C show schematic views of an ultraviolet irradiation device 100 according to the present embodiment. FIG. 1A is an external view illustrating a state in which the ultraviolet irradiation device 100 is attached to a work area, and FIG. 1B is a cross-sectional schematic view illustrating an internal configuration of the ultraviolet irradiation device 100. FIG. 1C is a timing chart showing an example of ultraviolet irradiation (ON) and non-irradiation (OFF) of the ultraviolet irradiation device 100. Note that part of the configuration is omitted as appropriate in FIGS. 1A-1C and the following figures in order to simplify the figures. Moreover, dimensions, shapes, thicknesses, etc. of components will be depicted in an exaggerated manner as appropriate in FIGS. 1A-1C and the following figures.

Referring to FIGS. 1A and 1B, the ultraviolet irradiation device 100 of the present embodiment is configured to disinfect a space, equipment, and the like in an area S to be disinfected by irradiating the area S to be disinfected with ultraviolet (indicated by broken lines in FIG. 1A for the sake of convenience). The ultraviolet irradiation device 100 includes a case 111, an ultraviolet irradiation unit 112 provided in the case 111, a drive control unit 113, a condensing unit 114, and a detection unit 115.

Note that the term “bacteria” to be disinfected as used in the description of the present embodiment is a generic term for bacteria (cells of germs, microorganisms, and viruses) mainly harmful to the human body (animals). The term “disinfection” by ultraviolet is defined as inactivating bacteria so as not to grow any more by directly acting on deoxyribonucleic acid (hereinafter, referred to as “DNA”) of the bacteria by light energy. Disinfection by ultraviolet refers to sterilization treatment of less than the 10⁻⁶ sterility level.

The area S to be disinfected is a working space into which a worker can enter or from which the worker can leave, and the working space is expected (required) to keep a predetermined level of cleanliness by controlling the number of bacteria, for example. An example of the area S to be disinfected in the present embodiment is an internal space of an operating room in a hospital and those found therein. Specific examples of the area S to be disinfected include the ceiling, floor, walls, room space (the air in the room), and equipment (its outer surfaces) disposed in the room. The area S to be disinfected in this case may include a surgical site of a patient having surgery.

The ultraviolet irradiation unit 112 is a unit capable of outputting ultraviolet (UV) having a predetermined dominant wavelength. More specifically, the ultraviolet irradiation unit 112 can output a wavelength in a short-wavelength (near-ultraviolet) UVC region of ultraviolet (ultraviolet light). Thus, the ultraviolet irradiation unit 112 is a UV light source having an ability to inactivate bacteria by directly destroying deoxyribonucleic acid (DNA) of the bacteria (germs) by its light energy.

Specifically, the ultraviolet irradiation unit 112 is a straight-tube low-pressure mercury lamp (low-pressure UV lamp), for example, and also a discharge lamp (metal vapor discharge lamp) utilizing emission of arc discharge in mercury vapor, which has an internal pressure (mercury vapor pressure) smaller than or equal to 100 Pa when lighted. The low-pressure mercury lamp (low-pressure UV lamp) 112 has a dominant wavelength of 250 nm to 260 nm, preferably 253 nm to 255 nm, and more preferably 253.5 nm to 254 nm (for example, 253.7 nm), for example.

The low-pressure mercury lamp 112 includes, at least anteriorly in an ultraviolet emitting direction, an inhibition unit 116 that inhibits generation of ozone. The inhibition unit 116 in this example is a lamp sleeve 116 of the low-pressure mercury lamp 112, which is made of quartz glass. Far-infrared light having a wavelength of 184.9 nm of ultraviolet wavelengths reacts with oxygen in the air, and thereby produces ozone. The low-pressure mercury lamp 112 in the present embodiment cuts a wavelength of 184.9 nm that produces ozone from the emitted ultraviolet by causing the emitted ultraviolet to pass through the inhibition unit (quartz glass lamp sleeve) 116.

Although FIG. 1B shows an example in which the low-pressure mercury lamp 112 is provided in a lower part of the exterior of the case 111, a lower surface of the case 111 may be constituted of a transparent member and the low-pressure mercury lamp 112 may be accommodated (attached) inside the case 111.

The condensing unit 114 that condenses ultraviolet irradiation directions into a predetermined direction is provided around or near the low-pressure mercury lamp 112. The condensing unit 114 is a member having a function to narrow down (focus) light such as a reflector, a screen, or a lens, for example.

In the ultraviolet irradiation device 100 of the present embodiment, a plurality of the above-described low-pressure mercury lamps 112 are provided in the case 111 and the low-pressure mercury lamps 112 are disposed so as to be spaced apart from each other at a predetermined interval.

The drive control unit 113 includes, for example, a drive power source 113A and a control unit 113B. The drive control unit 113 performs time control of ultraviolet irradiation and non-irradiation by the ultraviolet irradiation unit 112 as shown in FIG. 1C on the basis of time necessary to disinfect the area S to be disinfected during operation and bacterial growth time after the disinfection. The drive power source 113A is connected to a power source, or the like, of the area S to be disinfected, and efficiently turns the plurality of low-pressure mercury lamps 112 on and off individually. The control unit 113B includes a control circuit comprising a CPU, a RAM, a ROM, and the like, and executes various control operations. The CPU is what is called a central processing unit, and executes various programs including a program for controlling the turning-on and turning-off of the low-pressure mercury lamps 112 to implement various functions. The RAM is used as a workspace for the CPU. The ROM stores a basic operating system and programs to be executed in the CPU.

In general, ultraviolet irradiation at a level capable of disinfection is harmful to the human body. If a degree of contamination is high, the ultraviolet irradiation device 100 of the present embodiment performs disinfection treatment by applying ultraviolet at a level capable of disinfecting a predetermined kind of bacteria under conditions that the area S to be disinfected is not in operation, i.e., the area S to be disinfected is without people and not in use.

If a degree of contamination is low (a certain level of cleanliness is being maintained) after the above-described disinfection treatment has been performed once, bacterial growth in the area S to be disinfected can be suppressed by performing ultraviolet irradiation for a short amount of time even during operation (in use) of the area S to be disinfected without affecting the human body while recognizing the circumstances with regard to the presence of a person.

Referring to FIG. 1C, the drive control unit 113 controls the low-pressure mercury lamp 112 so that the low-pressure mercury lamp 112 is turned on in the first disinfection treatment (for example, before the operation (use) of the area S to be disinfected, if a degree of contamination is high) of a disinfection treatment process SE, the low-pressure mercury lamp 112 is turned off after ultraviolet is applied during first time (period) T1, and the ultraviolet non-irradiation state is maintained during second time (period) T2, for example.

The first time T1 in this case is an amount of time during which the first disinfection treatment can be performed, and the second time T2 is an amount of time during which the growth of the predetermined kind of bacteria after the passage of the first time can be suppressed. The second time T2 is longer than the first time T1.

Since the contaminated state has been reduced to some extent after the first disinfection treatment, the growth of the predetermined kind of bacteria is suppressed by briefly applying ultraviolet for a short amount of time during the operation (use) of the area S to be disinfected. That is, the drive control unit 113 turns on the low-pressure mercury lamp 112 again after the passage of the second time t2, and ultraviolet is applied again during third time (period) T3. Thereafter, the ultraviolet irradiation unit 112 is controlled so that the low-pressure mercury lamp 112 is turned off again and the non-irradiation state is maintained again during fourth time (period) T4.

The third time T3 in this case is a short amount of time during which bacteria that have increased during the period from the start to the end of the second time T2 (during the T2 period) can be disinfected after the passage of the second time T2, and the fourth time T4 is an amount of time during which bacterial growth after the passage of the third time T3 can be suppressed. The fourth time T4 is longer than the third time T3. The third time T3 in this case is shorter than the first time T1. Thereafter, turning on the low-pressure mercury lamp 112 for the third time T3 and turning off the low-pressure mercury lamp 112 for the fourth time T4 are repeated on the basis of using time of the area S to be disinfected.

The drive control unit 113 can individually control the turning-on and turning-off of the plurality of low-pressure mercury lamps 112 in the case 111. Thus, the turning-on, blinking, or turning-off of the plurality of low-pressure mercury lamps 112 can be achieved in a manner set as desired such as sequentially turning on the plurality of low-pressure mercury lamps 112, rotating the plurality of low-pressure mercury lamps 112 in a circular motion, or individually turning on the plurality of low-pressure mercury lamps 112 in a random manner, for example. This enables ultraviolet to be applied without creating a shadow (an unirradiated portion) to the area S to be disinfected (or a specific object to be disinfected that is present in the area), i.e., thoroughly (the shadow that blocks out ultraviolet can be minimized) during lighting (during ultraviolet irradiation).

The ultraviolet irradiation device 100 further includes the detection unit (human detection sensor) 115 configured to detect the presence or absence of a person at least in an ultraviolet irradiation region in the area S to be disinfected. When the human detection sensor 115 detects the presence of a person, the drive control unit 113 turns the low-pressure mercury lamp 112 into a non-irradiation state.

The human detection sensor 115 is integrally attached to the interior or exterior of the case 111 of the ultraviolet irradiation device 100. Alternatively, the human detection sensor 115 may be provided separately from the ultraviolet irradiation device 100, and may be electrically connected to the drive control unit 113 so that the human detection sensor 115 can send or receive a signal to or from the drive control unit 113. The human detection sensor 115 may be configured so that its function can be manually activated or deactivated (forcibly activated or deactivated).

With such a configuration, the ultraviolet irradiation device 100 of the present embodiment performs time control of irradiation and non-irradiation with ultraviolet having a predetermined dominant wavelength on the basis of time necessary to disinfect the area S to be disinfected during operation to fulfill its originally intended function (for example, during surgery in the case of an operating room) and bacterial growth time after the disinfection.

### Ultraviolet Irradiation Method

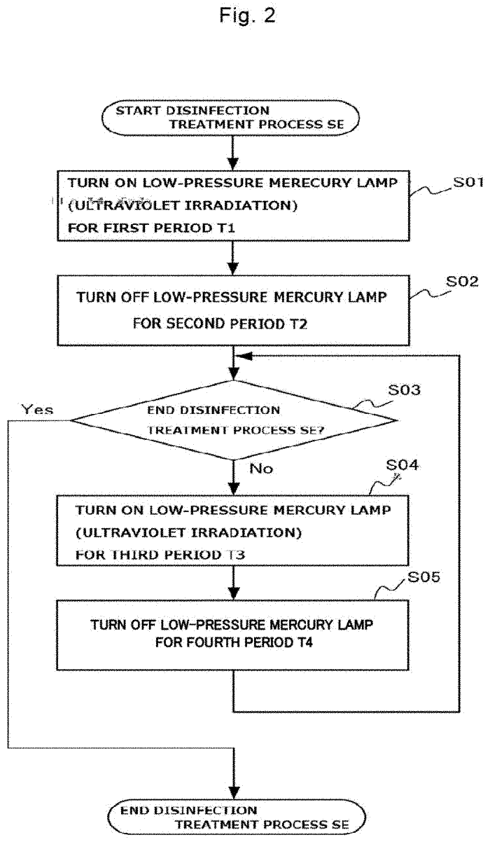

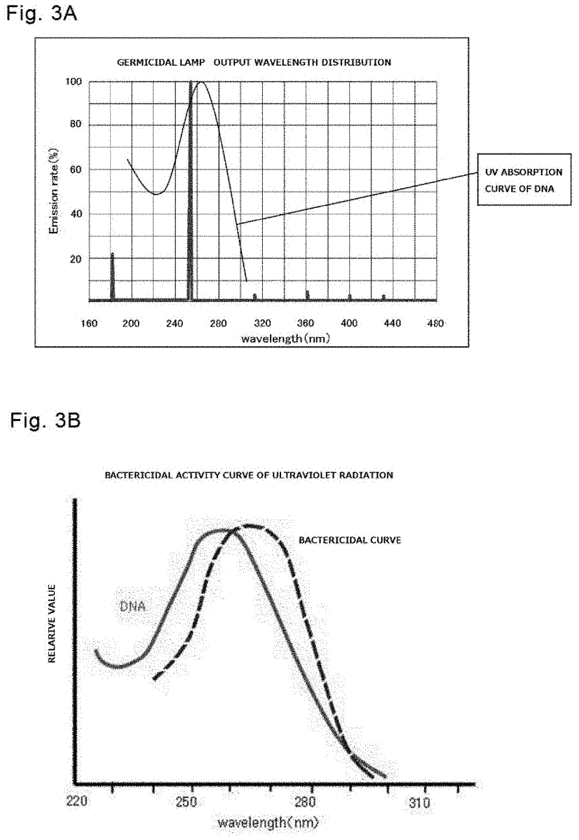

An ultraviolet irradiation method (a method of ultraviolet irradiation treatment) of the present embodiment will be described with reference to FIG. 2. The ultraviolet irradiation method of the present embodiment is for applying disinfection treatment to the area S to be disinfected by performing time control of irradiation and non-irradiation with ultraviolet having a predetermined dominant wavelength on the basis of time necessary to disinfect the area S to be disinfected during operation to fulfill its originally intended function (for example, during surgery in the case of an operating room) and bacterial growth time after the disinfection. The ultraviolet irradiation method of the present embodiment is implemented by the ultraviolet irradiation device 100 shown in FIG. 1, for example.

FIG. 2 is a flow diagram illustrating a procedure of an ultraviolet irradiation (disinfection) treatment process SE (see FIG. 1C).

First, in step S01, ultraviolet is applied during the first time (period) T1 in the first disinfection treatment (for example, after the area S to be disinfected (an operating room) is used, or immediately before its use) in the area S to be disinfected. The first time T1 is the shortest possible period among amounts of time capable of achieving the first disinfection (the removal of bacteria under the condition of a high degree of contamination) in the area S to be disinfected.

Subsequently, in step S02, the ultraviolet non-irradiation state is maintained during the second time (period) T2 after the passage of the first time T1 (step S02). The second time T2 is the longest possible period among amounts of time during which the growth of the predetermined kind of bacteria after the passage of the first time T1 can be suppressed.

After the passage of the second time T2, the process proceeds to step S03 and determines if the disinfection treatment process SE is ended. If the area S to be disinfected is continuously used (when the surgery is ongoing, for example, in the case of No in step S03), the process proceeds to step S04 and subsequent steps. Ultraviolet irradiation and non-irradiation are continuously repeated without affecting the human body while recognizing the circumstances with regard to the presence of a person.

In step S04, ultraviolet is applied again during the third time (period) T3 after the passage of the second time T2 to sterilize the bacteria that have increased during the period from the start to the end of the second time T2 (during the T2 period. The third time T3 is the shortest possible period among amounts of time during which increased predetermined target bacteria can be disinfected after the passage of the second time T2 (after the first disinfection treatment is ended). The third time T3 is shorter than the first time T1.

In step S05, non-irradiation is maintained again during the fourth time (period) T4 after the passage of the third time T3. The fourth time T4 is the longest possible period among amounts of time during which the growth of the predetermined target bacteria after the passage of the third time T3 can be suppressed.

Thereafter, the process returns to step S03, and steps S04 and S05 are appropriately repeated on the basis of the use (operating time) of the area S to be disinfected.

FIGS. 1C and 2 show, by way of example, the case in which the third period T3 is constant over the repeated turning-on operations and the fourth time T4 is constant over the repeated turning-off operations. The third period T3, however, may be set so as to become shorter gradually toward the end of the disinfection treatment process SE, and the fourth time T4 may be set so as to become longer gradually (longer than the immediately preceding third time T3) toward the end of the disinfection treatment process SE. If the number of bacteria is increased at some midpoint, the third time T3 may be set so as to be longer than the previous third time T3 and the fourth time T4 may be set so as to be shorter than the previous fourth time T4 (but longer than the immediately preceding third time T3).

In accordance with the contaminated conditions of the area S to be disinfected, the disinfection treatment process SE may be ended after steps S01 to S02 are performed.

The presence or absence of a person at least in the ultraviolet irradiation region in the area S to be disinfected is monitored constantly during the ultraviolet irradiation treatment (disinfection treatment). When the presence of a person is detected, ultraviolet irradiation is stopped. When the absence of a person is detected, ultraviolet is applied again.

In an area (the area S to be disinfected) in which the number of bacteria is controlled and a predetermined level of cleanliness needs to be maintained (a low contaminated state, a (substantially) aseptic state), disinfection treatment via, for example, formalin fumigation, EOG sterilization, or cleaning with a disinfectant (disinfection treatment according to a conventional method) has been conventionally performed.

If the area S to be disinfected is an operating room, a treatment room, an intensive care unit (ICU), or the like in a hospital, for example, an operating table, a bed, a shadowless lamp, an anesthesia apparatus, a patient surveillance monitor device, an endoscope television device, treatment tools, etc. are arranged in the area S to be disinfected as medical equipment items. In the area S to be disinfected, the number of bacteria needs to be regulated not only as a room (space) such as an operating room, a treatment room, or an ICU, but also as individual instruments.

Disinfection treatment according to such conventional methods, however, requires much time and efforts as well as a high cost, thus resulting in a reduced operating rate of a facility or equipment, i.e., the area S to be disinfected. Due to large burdens in terms of manpower and expenses, it is hard to perform such disinfection treatment frequently.

Disinfection treatment by ultraviolet (UV) irradiation has been also performed as a method that can be implemented relatively easily in conjunction with the above-described conventional disinfection treatment.

In the case of medical tools, for example, a variety of small steel products such as scissors or forceps are reused after being cleaned with an ultrasonic cleaning method, for example, and then subjected to a process such as autoclave sterilization or EOG sterilization in accordance with cleaning assessment guidelines (Japanese Society of Medical Instrumentation, Certification committee of sterilization technician, 2012). For their storage, a hygienic control device such as a UV disinfection cabinet may be employed if desired in order to take measures against secondary contamination.

The disinfection treatment by ultraviolet irradiation, however, raises problems such as harmful effects on the human body including the generation of ozone, and deterioration in equipment subjected to the irradiation. In an operating room or a treatment room, for example, medical workers frequently come into the room. Thus, it is generally difficult to perform the disinfection treatment by ultraviolet irradiation in consideration of effects on their health.

Equipment items disposed in an operating room or a treatment room are preferably disposed around a patient and medical workers from the standpoint of work efficiency. Thus, if ultraviolet is applied during the use of these items, the medical workers and the patient are in danger of being adversely affected.

For this reason, ultraviolet irradiation for disinfection treatment has been conventionally used only in a limited way at a limited place such as direct irradiation to the inside of an electronic device in which ultraviolet is never irradiated to the human body, or parts of surgery instruments or haircutting instruments, or irradiation to the inside of a room without people.

With regard to medical equipment including an operating table, a shadowless lamp, an anesthesia apparatus, a patient surveillance monitor system, and an endoscope television device, the number of bacteria needs to be regulated as individual devices as described above. Due to large size of such equipment, however, disassembling each device into parts and performing sanitization treatment on these parts using the above-described sterilizer every time are unpractical both physically and in terms of temporal limitations. Thus, as practical treatment, spraying alcohol and wiping out the alcohol before and after a procedure is a principal measure being taken. To apply perfect sterilization and sanitization treatment to the surface of each of these tools creates a large burden on workers. This is a major factor for hindering improvement in efficiency.

Furthermore, even after the disinfection treatment according to the conventional method as described above is performed, it is impossible to avoid bacteria being attached to a worker or the like from falling in the area S to be disinfected when the worker or the like enters and leaves the area S to be disinfected.

If the area S to be disinfected is an operating room, for example, medical workers such as doctors and nurses, patients, etc. enter and leave the operating room after the disinfection treatment according to the conventional method is performed. Thus, the intrusion of bacteria into the operating room is inevitable in a precise sense during work (during surgery) actually requiring a (substantially) aseptic state. Even so, if the bacteria having intruded after the disinfection treatment are allowed to grow, a big trouble such as hospital infection occurs in the worst case scenario.

It is therefore significantly important and efficient to perform disinfection (bacteria elimination) treatment with a high frequency without placing a long time interval between operations after performing the disinfection treatment according to the conventional method in terms of preventing infection spread. The disinfection treatment according to the conventional method, however, cannot be performed during the use of the area S to be disinfected, for example, during surgery.

Thus, it is difficult to perform the disinfection treatment according to the conventional methods, including spraying and wiping out alcohol, and the disinfection treatment by UV irradiation, with a high frequency (especially also during the operation of the area S to be disinfected).

As a result of earnest efforts to solve the aforementioned problem, the applicant of the present application has found out that the length of death time of a predetermined kind of bacteria by ultraviolet is short relative to time taken for a bacterial cell to double by division, i.e., the predetermined kind of bacteria is killed in a short amount of time when irradiated with ultraviolet, and then even when the ultraviolet irradiation is stopped for an amount of time sufficiently longer than the death time, the growth of the bacteria can be suppressed for a certain period of time (its details will be described later).

In view of the above, the applicant of the present application has arrived at the ultraviolet irradiation device 100 that quantitatively knows the number of a predetermined kind of bacteria in the area S to be disinfected, disinfection time of the bacteria by ultraviolet irradiation, and growth time of the bacteria, and repeats, after the bacteria are disinfected once, a cycle of applying ultraviolet again before the bacteria grow by utilizing a difference between the disinfection time and the growth time.

With such an ultraviolet irradiation device 100, the growth of the predetermined kind of bacteria can be suppressed with a limited amount of ultraviolet irradiation without significantly reducing the operating rate of work in the area S to be disinfected.

Furthermore, by incorporating the human detection sensor 115 for detecting whether a person is present in the area S to be disinfected into the ultraviolet irradiation device 100, a period during which ultraviolet irradiation is interrupted by the presence of a person is appropriately inserted into the cycle of ultraviolet irradiation for disinfection and growth suppression. Consequently, ultraviolet irradiation timing can be controlled in accordance with the circumstances with regard to the presence of a person even during the operating time of the area S to be disinfected in the daytime instead of just irradiating the area S to be disinfected with ultraviolet mainly while nobody is present (for example, during the night). Thus, the area S to be disinfected can be utilized while maintaining the conditions for suppressing bacterial growth.

A light source having a high bactericidal power, providing less damage such as the generation of ozone to the human body, and capable of reducing deterioration in the area S to be disinfected (the ceiling, the walls, the floor, (the materials of) the equipment items, etc.) as much as possible is preferably used as a light source of the ultraviolet irradiation device 100.

In view of the above, the plurality of low-pressure mercury lamps 112 capable of individually controlling its turning-on and turning-off operations are provided in the ultraviolet irradiation device 100, and the ultraviolet irradiation devices 100 are disposed at required positions such as the ceiling, wall surface, post, and illumination fixture of a room at required intervals. The ultraviolet irradiation devices 100 are configured to apply ultraviolet with a limited essential (minimum) amount of ultraviolet irradiation at essential timing. The ultraviolet irradiation devices 100 can apply ultraviolet thoroughly in the area S to be disinfected with such an essential irradiation amount.

As previously mentioned, irradiation (turning-on and turning-off) control is performed in the ultraviolet irradiation device 100 of the present embodiment at timing determined on the basis of the growth and death behaviors of the predetermined kind of bacteria. Timing for suppressing the growth of the predetermined kind of bacteria, however, is determined on the basis of the wavelength and irradiation intensity of ultraviolet to be used, the growth rate of the bacteria, and the number of bacteria (bioburden) present in the area S to be disinfected. That is, although such timing cannot be uniquely defined, ultraviolet irradiation conditions (irradiation and non-irradiation timing) can be determined by conducting a falling bacteria test for each room, and performing validations in combination with a kind of bacteria, the number of the bacteria, the allowable number of the bacteria, the capacity of the room, an amount of staying time, etc.

As just described, in the case of a high degree of contamination, the ultraviolet irradiation device 100 of the present embodiment performs disinfection treatment via ultraviolet irradiation while nobody is present (for example, before the operation of the area S to be disinfected). After that, the ultraviolet irradiation device 100 of the present embodiment suppresses the growth of bacteria increased, for example, by workers going in and out during the operation (work) of the area S to be disinfected. This can prevent deterioration in the degree of cleanliness during work in a room ensured to have cleanliness by being subjected to disinfection treatment before the work, for example.

Thus, the number of bacteria can be regulated not only as a room such as an operating room, a treatment room, or an ICU, but also for individual equipment items disposed in such a room.

In combination with the ultraviolet irradiation device 100 of the present embodiment, disinfection treatment via formalin fumigation, or EOG sterilization, for example, may be employed at timing in the absence of a person as is conventionally done. In this case, the number of such conventional disinfection treatment operations can also be reduced.

### Inactivation Treatment of Bacteria by Ultraviolet Irradiation

Inactivating bacteria by ultraviolet irradiation, a difference between the disinfection time of the bacteria by ultraviolet irradiation and the growth time of the bacteria, which has been found out by the applicant of the present application, and the principles of the ultraviolet irradiation device 100 utilizing such a difference will be described below.

Light energy having a wavelength shorter than 380 nm in optical radiation is referred to as ultraviolet radiation, and the ultraviolet radiation is known to exert various actions on substances and living organisms. As a characteristic of light, energy (kJ/mol) of light increases as the wavelength of the light becomes shorter. In particular, ultraviolet having a wavelength in the UVC region (100 nm to 280 nm) can degrade nucleic acid molecules or protein in a living organism.

Single bond of carbon atoms, on the other hand, do not absorb light with wavelengths longer than 230 nm, thus obtaining no chemical change. A nucleic acid change requires photon to be absorbed into double bonds contained in the nucleic acid. In principle, the inactivation of a microorganism occurs as follows: light energy having a peak wavelength of 260 nm is absorbed by bases of DNAs and ribonucleic acids (hereinafter, referred to as “RNA”) governing genetic information in the nucleus of a cell in a living organism, so that absorption causes the dimerization of thymine, etc., resulting in inhibition of further replication at the time of cell division.

On the basis of these facts, ultraviolet (UV) lamps capable of producing an output in the ultraviolet short-wavelength UVC region have been widely used in the fields of food, packaging films, water treatment, and disinfection treatment for airborne bacteria and falling bacteria in a space, for example, as energy capable of efficiently performing disinfection (inactivation of cells of bacteria or viruses) to improve hygienic control mainly for food and medical industry uses.

As an example of an ultraviolet lamp capable of outputting light energy in the UVC region, the ultraviolet irradiation unit 112 of the present embodiment employs a mercury lamp (the low-pressure mercury lamp 112) including mercury in its discharge tube.

FIGS. 3A and 3B show diagrams representing states of DNA inactivated by ultraviolet. FIG. 3A is a diagram showing an ultraviolet (UV) absorption curve of DNA superimposed on an output wavelength (spectral energy) distribution of the low-pressure mercury lamp 112. The UV absorption curve as used herein shows relative values of UV absorption rates of DNA according to UV wavelengths wherein the absorption rate of DNA at a UV wavelength of 260 nm is assumed to be 100. The vertical axis of FIG. 3A represents a relative value of a UV absorption rate, whereas the horizontal axis thereof represents a UV wavelength. FIG. 3B shows a UV absorption curve of DNA (solid line) and a bactericidal activity curve by UV (broken line). The bactericidal activity curve as used herein shows relative values of bactericidal rates of DNA according to UV wavelengths wherein the bactericidal (inactivation) rate of DNA at a UV wavelength of 260 nm is assumed to be 100. The vertical axis of FIG. 3B represents a relative value of a bactericidal rate, whereas the horizontal axis thereof represents a UV wavelength (nm).

As shown in FIG. 3A, the low-pressure mercury lamp can obtain, as a dominant wavelength, an emission line at 253.7 nm emitted when electrons collide with mercury in the discharge tube. A spectrum absorbed by DNA of a living organism (the same applies to RNA) extends over a wavelength region having its center around 260 nm. As previously mentioned, although the bactericidal activity by ultraviolet radiation occurs as a result of damaging DNA, the bactericidal activity curve showing the bactericidal effect approximately coincides with the UV absorption curve of DNA as shown in FIG. 3B. The reason for this is as follows: pyrimidine groups continuously present in DNA are dimerized by absorbing light in this wavelength region, so that dimerization damages genetic code, and thus, the cell loses its ability to differentiate and becomes inactivated.

That is, a high level of sanitization (the inactivation of cells) treatment can be performed by efficiently irradiating target bacteria with light of energy of 253.7 nm outputted from the low-pressure mercury lamp.

A fluorescent lamp causes such light with energy of 253.7 nm to hit a fluorescent material applied to an inner wall of a glass arc tube to convert it into visible light and utilizes the visible light as illumination. In the case of a germicidal lamp, however, UV transmitting glass that can efficiently transmit an ultraviolet having short wavelengths and quartz glass having an even higher transmitting property are used. As a mercury lamp of the same kind, there is a high-pressure mercury lamp (sometimes referred to as a medium-pressure mercury lamp used for an industrial purpose) capable of obtaining high intensity and mainly used as a street light. The high-pressure mercury lamp, however, emits many heat rays too. Thus, a low-pressure mercury lamp capable of reducing heat rays and capable of efficiently obtaining light with a wavelength of 253.7 nm is employed as the ultraviolet irradiation unit 112 in the present embodiment.

A UV light with a wavelength of 184.9 nm causes a reaction between oxygen to produce ozone. Thus, such the UV light has a risk of deteriorating the components or adversely affecting the human body. For this reason, the low-pressure mercury lamp (ultraviolet irradiation unit) 112 of the present embodiment includes the inhibition unit 116 capable of cutting the light (component) with a wavelength of 184.9 nm in order to inhibit the generation of ozone by ultraviolet irradiated from the low-pressure mercury lamp into the air. Specifically, the inhibition unit 116 is a lamp sleeve made of quartz glass. Note that an inhibition unit 116 made of quartz glass may be separately provided to the front surface of the ultraviolet irradiation unit 112 in the ultraviolet emitting direction.

The disinfection (inactivation) treatment of bacteria by UV is disadvantageous in that the treatment cannot be performed unless a prescribed amount of light is applied. The disinfection (inactivation) treatment of bacteria by UV, however, is advantageous in that effective treatment can be performed on any kind of bacteria since no drug-resistant strain of bacteria, which becomes a problem in disinfection treatment methods via drugs or heat, for example, emerges.

Although the low-pressure mercury lamp inconsiderably outputs light (components) with wavelengths of not shorter than 310 nm in FIG. 3A, any of these wavelengths has an absorption rate of DNA lower than or equal to about 5%. These wavelengths can be therefore considered substantially negligible from the standpoint of bactericidal activity.

The bactericidal activity by ultraviolet is herein determined by a cumulative amount of light (cumulative irradiation amount) (μj/cm² (mJ/cm²)) for light energy in a bactericidal wavelength band given to DNA of bacteria (cells). The cumulative amount of light is a product of UV intensity (UV radiation intensity (illuminance)) per a unit area (μw/cm² (mw/cm²)) and irradiation time (sec) (Expression 1).

Cumulative amount of light (μj/cm²)=UV illuminance (μW/cm²)×time (sec) (Expression 1)

The disinfection treatment by ultraviolet is effective to all kinds of bacteria. Nevertheless, since tolerance (sensitivity) to ultraviolet varies among kinds of bacteria, a required ultraviolet irradiation amount is determined for each kind of bacteria to be disinfected on the basis of its disinfection treatment indicator.

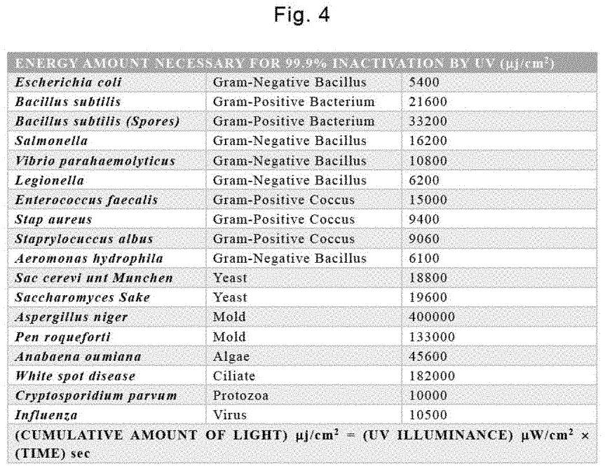

FIG. 4 is a table showing, for each kind of bacteria, an example of a cumulative amount of light necessary to inactivate 99.9% or more of bacteria when UV in a range of 267 nm to 287 nm is applied thereto (Reference: The illuminating Engineering Society (IES) lighting handbook).

Referring to FIG. 4, a cumulative amount of light necessary to disinfect 99.9% or more of Bacillus subtilis spores, which are used as a disinfection reference indicator for food, is 33,200 (μJ/cm²), and a cumulative amount of light necessary to disinfect 99.9% or more of influenza viruses is 10,500 (μJ/cm²), for example. That is, on the basis of these indicator values, a cumulative amount of light from the low-pressure mercury lamp 112 is set according to a kind of bacteria to be disinfected.

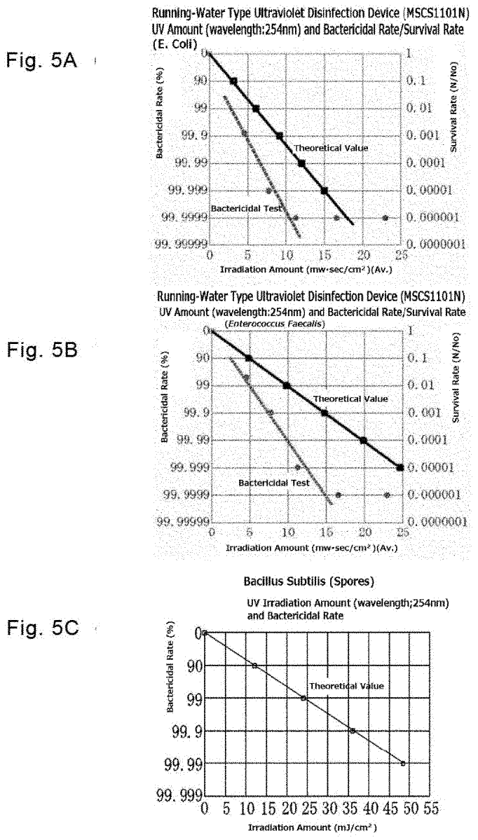

FIG. 5 shows graphs each representing bactericidal rates of bacteria by UV irradiation. The vertical axes thereof represent a bactericidal rate (%) and a survival rate (N/NO), whereas the horizontal axis thereof represents an average value (mw·sec/cm²) of a UV irradiation amount (illuminance). FIG. 5A is a graph of Escherichia coli, FIG. 5B is a graph of Enterococcus faecalis, and FIG. 5C is a graph of Bacillus subtilis. The solid line in each of these figures represents known theoretical values. The broken lines in FIGS. 5A and 5B each show plotted results of a bactericidal test actually performed by irradiating bacteria with ultraviolet using the low-pressure mercury lamp 112 of the present embodiment. FIGS. 5A to 5C each represent that a higher level of bactericidal effect can be obtained toward the lower end of the vertical axis of the graph and ability to kill bacteria increases by an order of magnitude for each row.

### Test Conditions/Escherichia Coli

Test bacteria were inoculated into an SCDB culture medium, and then subjected to shaking culture at 35° C.±1° C. for 18 to 20 hours. The cultured bacterial cells were suspended in purified water so that the number of bacteria per 1 mL was about 10¹⁰ to prepare test bacteria liquid.

In addition, 100 mL of the test bacteria liquid was added to, and mixed with, about 500 L of raw water to prepare a test liquid. The test liquid was passed through the low-pressure mercury lamp 112 under the conditions of flow rates of 71 L/min, 95 L/min, 142 L/min, 213 L/min, and 370 L/min, and the passed-through water was collected. Subsequently, the number of bacteria in the test liquid before passing through the low-pressure mercury lamp 112 and the number of germs in the passed-through water were measured.

These measurements were obtained according to a pour plate culture method (cultured for 24 hours at 35° C.±1° C.) using an SA culture medium.

### Test Conditions/Enterococcus Faecalis

Test bacteria were inoculated into an SCDB culture medium, and then subjected to shaking culture at 35° C.±1° C. for 18 to 20 hours. The cultured bacterial cells were suspended in purified water so that the number of bacteria per 1 mL was about 10¹⁰ to prepare a test bacteria liquid.

In addition, 100 mL of the test bacteria liquid was added to, and mixed with, about 500 L of raw water to prepare a test liquid. The test liquid was passed through the low-pressure mercury lamp 112 under the conditions of flow rates of 8.3 L/min, 17 L/min, and 33 L/min, and the passed-through water was collected. Subsequently, the collected passed-through water was stored at 20° C.±1° C. for 14 days. The number of germs and the number of Enterococcus faecalis in the test liquid before passing through the low-pressure mercury lamp 112, in the passed-through water immediately after being collected, and in the passed-through water stored at 20° C.±1° C. for 14 days were measured.

The number of germs was measured according to the pour plate culture method (cultured for 24 hours at 35° C.±1° C.) using an SA culture medium, or a membrane filter method (cultured for 24 hours at 35° C.±1° C.). The number of Enterococcus faecalis was measured according to the pour plate culture method (cultured for 48 hours at 35° C.±1° C.) using a KF culture medium, or the membrane filter method (cultured for 48 hours at 35° C.±1° C.).

Although the above-described bactericidal tests are running water tests, an object to be treated can be just changed from water to air. Bactericidal effect by UV becomes sufficient by ensuring an energy amount determined for each kind of bacteria, which is shown in FIG. 4. The same applies also to falling bacteria in the area S to be disinfected.

In the case of ultraviolet disinfection, a bactericidal rate is determined by the irradiation amount of light. Thus, an irradiation distance (distance from the UV light source) also affects the bactericidal rate, and the effect also varies significantly depending on the duration of irradiation time. In the case of the low-pressure mercury lamp 112 of the present embodiment shown in FIGS. 5A and 5B, for example, the irradiation distance from the furthest part (distance from the low-pressure mercury lamp 112 to the area S to be disinfected) is about 80 mm. If an object to be disinfected is a liquid material, the UV irradiation time can be set since a flow amount and a passage rate can be calculated from a factor such as a capacity of a treatment tank.

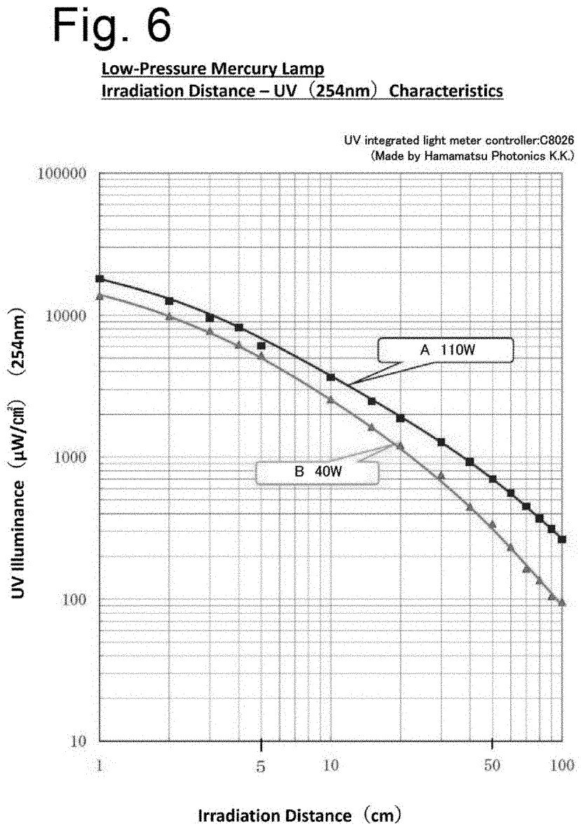

FIG. 6 is a graph showing a relationship between an irradiation distance of the low-pressure mercury lamp 112 of the present embodiment and UV illuminance (μW/cm²) at a wavelength of 254 nm, where A represents a 40-W lamp, and B represents a 110-W lamp.

According to FIG. 6, when the irradiation distance is 100 mm, for example, UV illuminance of about 2,500 μW/cm² can be obtained. Taking Escherichia coli as an example, 99.9% or more of Escherichia coli can be disinfected with an energy amount corresponding to a cumulative amount of light of 5,400 μJ/cm² according to FIG. 4. Therefore, it can be figured out that a sufficient level of bactericidal effect can be obtained by UV irradiation for 5400/2500=2.16 sec. Since UV theoretically decays inversely with the square of distance, disinfection at a rate of 99.9% or more can be achieved by UV irradiation for several tens of minutes if the irradiation distance is about 1 m.

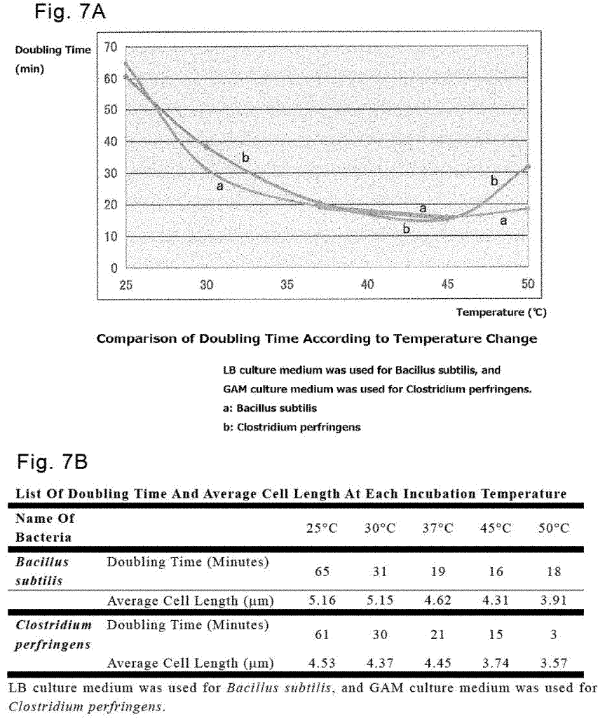

FIGS. 7A and 7B show diagrams showing comparison of doubling time (an amount of time taken for a bacterium to undergo cell division and thereby double (multiply)) varied according to changes in temperature between Bacillus subtilis and Clostridium perfringens (Reference: Hajime Okumura, “Comparative analysis of molecular mechanisms of cell-progression in Clostridium perfringens and Bacillus subtilis,” Master's thesis, Nara Institute of Science and Technology, Feb. 2, 2006). In FIG. 7A, the horizontal axis represents an incubation temperature (° C.), and the vertical axis represents doubling time (minutes). In FIG. 7A, “a” denotes Bacillus subtilis, and “b” denotes Clostridium perfringens. FIG. 7B is a list of doubling time and an average cell length at each incubation temperature. An LB culture medium was used for Bacillus subtilis, and a GAM culture medium was used for Clostridium perfringens.

In the case of Bacillus subtilis, for example, it can be seen with reference to FIG. 7B that 65 minutes are taken to double at 25° C. and 31 minutes are taken to double at 30° C.

Referring to FIG. 6, in the case of the 40-W low-pressure mercury lamp 112, for example, UV illuminance at an irradiation distance of 1 m is 100 μW/cm² (0.1 mW/cm²).

According to FIG. 5C, a cumulative amount of light of about 12 mJ/cm² is required to disinfect 90% of Bacillus subtilis (to reduce the risk of germ infection to one tenth). That is, by achieving UV illuminance of 0.1 mw/cm² when the irradiation distance is 1 m, infection risk of Bacillus subtilis can be reduced to one tenth by irradiation for 120 seconds (two minutes).

As already mentioned, FIG. 5C shows that a higher level of bactericidal effect can be obtained toward the lower end of the vertical axis of the graph and ability to kill bacteria increases by an order of magnitude for each row. In the above-described example of Bacillus subtilis, the infection risk is reduced to one tenth in two minutes, the infection risk is reduced to one hundredth in four minutes, the infection risk is reduced to one ten-thousandth in eight minutes, and the infection risk is reduced to one hundred-thousandth in 10 minutes.

From the foregoing, if the area S to be disinfected is initially irradiated with ultraviolet for 10 minutes by the low-pressure mercury lamp 112, for example, the infection risk of bacteria (Bacillus subtilis) in the area S to be disinfected is reduced to one hundred-thousandth. The doubling time of Bacillus subtilis, on the other hand, is about 20 to 60 minutes at room temperature according to FIG. 7. It can be said that increase in bacteria during such a period is negligible.

Once the number of bacteria in the area S to be disinfected is significantly reduced by initially performing disinfection treatment with expenditure of sufficient time, the number of bacteria introduced into the area S to be disinfected thereafter while being attached to a worker (medical worker), for example, is few. To disinfect such few bacteria, the UV irradiation amount (irradiation time) can also be reduced significantly as compared to the initial amount of time.

As described above, the applicant of the present application has focused on the point that an amount of time taken for a kind of bacteria to double (multiply) (20 to 60 minutes in the above-described example) is longer than an amount of time necessary to disinfect the kind of bacteria (about 10 minutes in the above-described example). The applicant of the present application has found out that disinfection treatment can be done efficiently and safely by performing ultraviolet irradiation utilizing such a time difference. The applicant of the present application also has found out that bacteria newly introduced after disinfection is initially performed with expenditure of a certain amount of time can be efficiently disinfected even during the use of the area S to be disinfected, for example, by performing UV irradiation while work is interrupted and workers are evacuated from the area S to be disinfected only for a short amount of time. On the basis of these findings, the applicant of the present application has been able to arrive at the ultraviolet irradiation device 100 of the present application.

The ultraviolet irradiation device 100 of the present application can perform time control of ultraviolet irradiation and non-irradiation by the low-pressure mercury lamp 112 on the basis of time necessary to disinfect the area S to be disinfected before or during operation and bacterial growth time after the disinfection.

Specifically, the low-pressure mercury lamp 112 can output ultraviolet in the UVC region (its dominant wavelength is about 254 nm) having a dominant wavelength with a high bactericidal power (capable of efficiently inactivating DNA). The low-pressure mercury lamp 112 can also reduce an amount of time necessary for disinfection, and can finely set and control the turning-on (ON) and turning-off (OFF) operations so as not to affect the human body.

More specifically, as shown in FIG. 1C, the ultraviolet irradiation device 100 is turned on initially (before the operation of the area S to be disinfected, the first disinfection treatment) to apply ultraviolet for a certain amount of time (the first time T1) enough to reduce the number of bacteria significantly, and then the ultraviolet irradiation device 100 is turned off for an amount of time (the second time T2) during which bacterial growth in the area S to be disinfected can be suppressed and that is longer than the first time T1. After that, turning on the ultraviolet irradiation device 100 for a short amount of time (the third time T3) during which a very small amount of bacteria increased in the area S to be disinfected can be disinfected, and turning off the ultraviolet irradiation device 100 for an amount of time (the fourth time T4) during which the growth of the bacteria can be suppressed and that is longer than the third time T3 are (repeatedly) performed. In this manner, the disinfection treatment can be performed efficiently and safely with an ultraviolet irradiation amount as small as possible even during the operation of the area S to be disinfected.

The ultraviolet irradiation device 100 may include a plurality of low-pressure mercury lamps 112 capable of individually controlling their turning-on and turning-off operations. While being lighted (during the disinfection treatment), the turning-on operations may be controlled by sequentially switching the plurality of low-pressure mercury lamps 112 so as not to create a shadow area to which no ultraviolet is applied.

The ultraviolet irradiation device 100 may include the condensing unit (function to narrow down the optical path) 114 such as a reflector, a screen, or a lens so that ultraviolet can be applied intensively to a particular region in the area S to be disinfected.

The ultraviolet irradiation device 100 may include the human detection sensor 115. Even while being lighted (during the disinfection treatment), the ultraviolet irradiation device 100 may be turned off or ultraviolet may be shielded by a shutter or a screen, for example, if the presence of a person is detected. In this manner, adverse effects on the human body may be avoided. Alternatively, an alarming sound (notification music or an alarm sound) may be outputted, for example, when the human detection sensor 115 detects the presence of a person.

With such a configuration, uncertainty in disinfection treatment can be improved, time and effort needed to control the number of bacteria in the area S to be disinfected and needed to conduct facility management can be reduced while ensuring safety to the human body, such as a worker (medical worker), and disinfection and the prevention of bacterial growth can be efficiently performed without significantly deteriorating the operating rate of the area S to be disinfected.

### Layout Example and Irradiation Control Example of Ultraviolet Irradiation Device 100

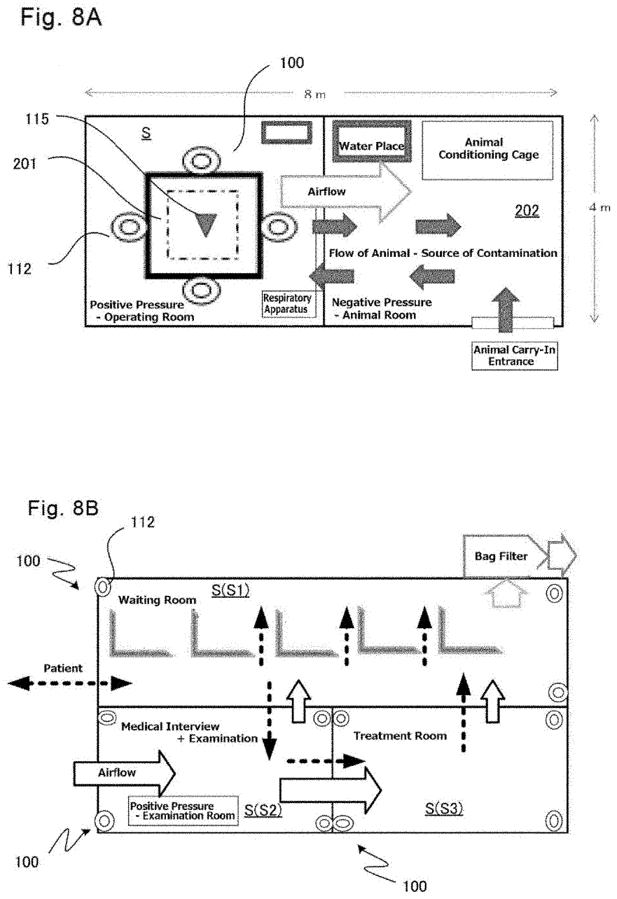

A layout example and an irradiation control example of the ultraviolet irradiation device 100 will be described next more specifically with reference to FIGS. 8A and 8B. FIGS. 8A and 8B show schematic top views each illustrating an example of the area (facility) S to be disinfected in which the ultraviolet irradiation device 100 of the present embodiment is disposed. FIG. 8A shows a case where the area S to be disinfected is an operating room for animal testing. FIG. 8B shows a case where the area S to be disinfected is a medical facility for accepting infected patients. Note that a main purpose of FIGS. 8A and 8B is to show layout examples of the ultraviolet irradiation units (low-pressure mercury lamps 112) in the ultraviolet irradiation device 100, and the illustration of the other configurations (other configurations for the case 111, the drive control unit 113, etc.) are therefore omitted.

Referring to FIG. 8A first, the area S to be disinfected is an operating room for animal testing, for example. For the dimensions of the room, the floor area is 32 (8 m×4 m) m², and the ceiling height is 2.5 m, for example. Water-resistant coating is applied to the floor, and water-resistant resin coating is applied to the ceiling. A high-efficiency particulate air (HEPA) filter (not shown) is provided in the center of the ceiling of the room. As equipment in the operating room S, a four-square-meter operating table 201 made of SUS and having a height of 0.7 m is disposed approximately in the center of the room.

The ultraviolet irradiation device 100 of the present embodiment is provided near the center of the ceiling. The ultraviolet irradiation device 100 includes four low-pressure mercury lamps 112 (40 W), for example. Each of the low-pressure mercury lamps 112 is suspended from the ceiling at a distance of 0.7 m from the ceiling. The human detection sensor 115 is disposed approximately in the center of the four low-pressure mercury lamps 112, for example, and an informing sound output unit (speaker) is also provided (not shown). An operating part of the ultraviolet irradiation device 100 is provided outside of the operating room S (anteroom (animal room) 202), for example.

The anteroom 202 is set to negative pressure and the operating room S is set to positive pressure so that airflow flows from the operating room S to the anteroom 202. An animal is brought in from an animal carry-in entrance of the anteroom, and moved to the operating room S through the anteroom. After surgery, the animal is transferred from the operating room S to the anteroom 202 (moved according to directions of small arrows).

The airflow flows in the direction of a large arrow from the positive-pressure operating room to the negative-pressure anteroom 202.

The output wavelength of the low-pressure mercury lamp 112 is the short-wavelength UVC region of ultraviolet light, and the ultraviolet light has the ability to inactivate germs by directly destroying DNA of the germs. Specifically, the lamp sleeve 116 comprises ozone free quartz capable of cutting light (component) with a wavelength of 184.9 nm, and the low-pressure mercury lamp 112 is configured to output only a wavelength (energy) of about 245 nm (253.7 nm).

In order to allow ultraviolet to be focused on an area (the operating table 201) requiring an aseptic state most, the low-pressure mercury lamps 112 are disposed above the operating table 201 so as to surround the operating table 201 and be separated from one another. The low-pressure mercury lamps 112 are turned on by an ON and OFF control program (part of the drive control unit 113) of the low-pressure mercury lamps set in consideration of a relationship between ultraviolet irradiation time necessary for disinfection and bacterial growth time.

Detecting the presence or absence of a person by the human detection sensor (see FIG. 1) and performing irradiation mainly during the absence of a person are incorporated into the above-described control program. In addition, the four low-pressure mercury lamps 112 are turned on and off while changing their positions, for example, rotating the low-pressure mercury lamps 112 or turning on the low-pressure mercury lamps 112 in a random manner, so as to reduce a shadowed area.

If the number of bacteria in the room is approximated by the number of particles, for example, the operating room S is comparable to class 100 (cleanliness class according to which the number of 0.5 μm particles is less than or equal to 100 in 1 ft³) of clean room standards (ISO 14644-1).

First of all, the number of bacteria in the operation area can be reduced before opening the chest (making an incision) of a patient (a laboratory animal) by using the ultraviolet irradiation device 100 of the present embodiment. Moreover, increase in the number of bacteria can be suppressed during the surgery by allowing workers to be evacuated only for a short amount of time in order to perform disinfection. Thus, the area S to be disinfected can be disinfected with the least possible interruption of the surgery (increasing the operating rate of the operating room). Moreover, after the completion of the surgery, the surgical field can be irradiated with ultraviolet for a short amount of time before the chest is closed (the incision is closed). By doing so, the chest can be closed with the existing probability of bacteria being lowered, and thus the postoperative infection probability can be lowered.

Specifically, an operation example of the area (operating room) S to be disinfected, and a disinfection treatment method for the area S to be disinfected are as follows.

Before the day preceding surgery, the operating room S is cleaned according to a known method, the walls, the floor, and the operating table are wiped with a conventional disinfectant, and a variety of surgical instruments disinfected according to a conventional method are prepared.

On the day of the surgery, a worker (an experimenter or an operator) wears a surgical gown (operating suit), a cap, a mask, surgical gloves, an eye goggle, etc. The worker then carries a subject animal in, administers an anesthetic to the animal, shaves the hair of the animal, broadly sanitizes the surgical site with an antiseptic solution, overlays a drape over the entire animal, and cuts off a portion of the drape corresponding to the surgical field. In this case, the number of bacteria is 0/cm² in the initial state by (completely) sanitizing a surgical field of 100 cm² with the antiseptic solution, for example. Subsequently (during the preparation, or during the surgery), falling bacteria increased in the area S to be disinfected are disinfected by the ultraviolet irradiation device 100.

All of the workers are evacuated to the outside of the operating room S (the anteroom 202), and the ultraviolet irradiation device 100 is turned on while nobody is present in the operating room S.

In the ultraviolet irradiation device 100, an informing sound (a warning melody) that informs the turning-on of the low-pressure mercury lamp 112 is outputted from the speaker while the low-pressure mercury lamp 112 is lighted. While the low-pressure mercury lamp 112 is unlit, the informing sound is stopped (alternatively, a safety-indicating melody that informs the turning-off of the low-pressure mercury lamp 112 is outputted).

The four germicidal lamps are sequentially turned on one by one in a clockwise direction, for example, so as not to create shadows. These germicidal lamps disinfect falling bacteria and airborne bacteria in the vicinity of the surgical field, and also prevent increase in bacteria.

The ultraviolet illuminance of the low-pressure mercury lamp 112 (40 W) in the ultraviolet irradiation device 100 is about 0.1 mw/cm² when the irradiation distance is 1 m (FIG. 6). Taking Bacillus subtilis (spores) in FIGS. 5C and 7 as an indicator bacterium, for example, a cumulative amount of ultraviolet necessary to reduce the infection risk in the area S to be disinfected to one tenth (a bactericidal rate of 99%) is 12 mJ/cm² (FIG. 5C).

That is, in the case of the low-pressure mercury lamp 112 having ultraviolet illuminance of about 0.1 mw/cm², irradiation time taken before the bactericidal rate reaches 99% is 120 seconds (12 (mJ/cm²)/0.1 (mw/cm²)).

Thus, according to the control program of the ultraviolet irradiation device 100 for the operating room S, if lighting is performed for 10 minutes, for example, in the first round (the first cycle) after the start of the work, the infection risk in the area S to be disinfected is reduced to one hundred-thousandth in the case of Bacillus subtilis (spores) (FIG. 5C). That is, even when Bacillus subtilis (spores) attached to, for example, the workers fall in the room or are introduced into the air immediately before the work or during the work, the infection risk is reduced to one hundred-thousandth.

An amount of time taken for Bacillus subtilis to undergo cell division and thereby double, on the other hand, is 30 to 60 minutes at room temperature (FIG. 7). Thus, increase in bacteria during such a period is negligible.

Specifically, in the first round (the first cycle) after the activation of the ultraviolet irradiation device 100, the low-pressure mercury lamp 112 is turned on, for example, for 10 minutes (the first time T1), and subsequently, the low-pressure mercury lamp 112 is turned off, for example, for sixty minutes (the second time T2). After that (in the second and subsequent cycles), turning on the low-pressure mercury lamp 112 for two minutes (the third time T3) and then turning off the low-pressure mercury lamp 112 for 28 minutes (the fourth time T4) are repeated until the end of the surgery. Note that an amount of time needed for the surgery is assumed to be six hours, for example.

In this example, since the entire body of the worker is shielded by the operating suit, etc., the worker is little affected by the exposure of ultraviolet. The laboratory animal is also little affected by the exposure of ultraviolet since the laboratory animal is covered with the drape except for the surgical field. Under the circumstances of eight-hour labor, however, the regulatory amount (limit dose of a cumulative amount of light) of ultraviolet in the UVC region is 3 mJ/cm². Thus, when the cumulative amount of light is 100 mJ/cm², irradiation time of 30 seconds is a limit. Thus, the safety of a worker is ensured by the human detection sensor and the informing sound so that the worker is prevented from being exposed to ultraviolet unintentionally while the low-pressure mercury lamp 112 is lighted.

In this example, the ultraviolet irradiation device 100 is activated immediately before the work in the area (operating room) S to be disinfected whose cleanliness has been ensured under normal (conventional) control, for example, by performing disinfection treatment according to the conventional method when the degree of contamination is high such as after surgery. Consequently, deterioration in cleanliness immediately before the work or during the work due to bacteria brought by the workers, the laboratory animal, etc., going into and out of the area S to be disinfected can be prevented from occurring.

After the completion of the surgery, the surgical field is sutured, and sanitized with a known antiseptic solution. Note that the surgical field may be intentionally irradiated in a concentrated manner and thereby disinfected by the low-pressure mercury lamp 112 only for a very short amount of time (for a short amount of time having no effects on the human body or the laboratory animal) before closing the chest or closing the abdomen.

After the surgery, clearance, cleaning, and disinfection treatment according to the conventional method are performed, the control program (on and off program) of the ultraviolet irradiation device 100 is adjusted. For example, the low-pressure mercury lamp 112 is turned on mainly during the non-operating period of the operating room S (such as scheduled night-time during which the operation is stopped). The low-pressure mercury lamp 112 is kept lighted as long as the human detection sensor detects the absence of a person. This can reduce the number of extensive disinfection treatment operations (according to the conventional method) to be performed as preparations for the upcoming use of the operating room S, or can improve the reliability of disinfection treatment performed in combination with the conventional method.

Once the human detection sensor 115 detects the presence of a person (including an animal), the low-pressure mercury lamp 112 is stopped. By reviewing the result, it becomes possible to take measures to obviate an unintentional future action (accident). Note that the operating room S may be an operating room of a general hospital to be used for the surgery of the human body.

Referring now to FIG. 8B, the area S to be disinfected is a medical facility for accepting infected patients, for example. This is a simplified (for example, prefabricated or modular) medical facility including three segments of a waiting room S1, a medical interview and examination room S2, and a treatment room S3 disposed adjacent to one another.

With regard to the dimensions of the rooms, the waiting room S1 has an area of 12 m², the medical interview and examination room S2 has an area of 6 m², and the treatment room S3 has an area of 6 m². The ceiling height of these rooms is 2.2 m, for example.

The waiting room S1 has negative pressure, the medical interview and examination room S2 has positive pressure, and the treatment room S3 has positive pressure. Thus, airflow in these rooms flows as indicated by hollow arrows. A filter (bag filter) is provided in a duct of the waiting room S1 so that no airflow in this room flows into the other rooms.

It is assumed that the waiting room S1 has the minimum equipment such as a sofa or a bulletin board (neither is shown).

The ultraviolet irradiation device 100 includes the four low-pressure mercury lamps 112 as with FIG. 8A, and each low-pressure mercury lamp 112 is disposed at each of the corner areas immediately under the ceiling. The human detection sensor is also provided although the illustration thereof is omitted.

Examples of equipment in the inside of the medical interview and examination room S2 include a desk and a chair for a doctor, a chair for a patient, and an electronic medical chart (none of these are shown).

The ultraviolet irradiation device 100 includes the four low-pressure mercury lamps 112 as with FIG. 8A, for example, and each low-pressure mercury lamp 112 is disposed at each of the corner areas immediately under the ceiling. The human detection sensor is also provided although the illustration thereof is omitted. (An air outlet of) a HEPA filter is provided at the ceiling.

A medical table for a nurse and a bed (neither is shown) are provided as equipment in the inside of the treatment room S3. The ultraviolet irradiation device 100 includes the four low-pressure mercury lamps 112 as with FIG. 8A, and each low-pressure mercury lamp 112 is disposed at each of the corner areas immediately under the ceiling. The human detection sensor is also provided although the illustration thereof is omitted. (An air outlet of) a HEPA filter is provided at the ceiling.

An operation example of the area (medical facility) S to be disinfected, and a disinfection treatment method for the area S to be disinfected are as follows.

In the waiting room S1, the medical interview and examination room S2, and the treatment room S3, before accepting a patient (in the first round), the low-pressure mercury lamps 112 are turned on, for example, for 10 minutes (the first time) and then turned off for 20 minutes (the second time). During such an unlit period, a patient is accepted.

Although a patient moves in the order of the waiting room S1, the medical interview and examination room S2, and the treatment room S3 as indicated by dashed arrows, the patient can move only when the low-pressure mercury lamps 112 in each room are unlit. For example, if a patient leaves one room (the medical interview and examination room S2, for example) (moves to the next room (the treatment room S3)) and the room becomes empty, the human detection sensor detects the absence of a person, and permits the next patient to enter the room (the medical interview and examination room S2, for example). In this case, a display unit that receives a signal from the human detection sensor and displays the permission (rejection) of the entry to the room, or a speaker to give spoken guidance, for example, may be provided at the door or near the entrance in each room to guide a patient. Alternatively, the doors of the rooms may be configured to open and close (or to lock and unlock) automatically in accordance with a detection result of the human detection sensor.

In this manner, disinfection treatment can be performed only for a period of time during which no patient is in the room, thereby reducing the risk of infection spread.

In this example, in each of the waiting room S1, the medical interview and examination room S2, and the treatment room S3, normal cleaning or planned (periodic) disinfection treatment according to the conventional method may be conducted during the absence of a patient. When an infected patient is accepted, the ultraviolet irradiation device 100 is turned on (in the first round) for 10 minutes before the acceptance, and then turned off for 20 to 30 minutes.

Note however that a patient (a source of infection) moves through the rooms, and the next patient similarly moves through the rooms in such a case. Thus, the turning-on and turning-off of the low-pressure mercury lamps in each room are controlled so as to reduce the risk of germ infection caused by the previous patient.

The ultraviolet illuminance of the low-pressure mercury lamp 112 in the ultraviolet irradiation device 100 is about 0.1 mw/cm² when the irradiation distance is 1 m, for example (FIG. 6). In order to reduce the risk of germ infection caused by the previous patient (patient as a source of infection) to one tenth, a necessary cumulative amount of ultraviolet is 12 mJ/cm² (FIG. 5C), and necessary irradiation time is 120 seconds. Thus, according to the control program of the ultraviolet irradiation device 100 in each of the medical interview and examination room S2 and the treatment room S3, even after the first (10 minutes) ultraviolet irradiation is performed, the next patient is accepted at a time interval of three minutes or more (two minutes of such three or more minutes is used for ultraviolet irradiation) after the patient left (a doctor and the like person also left).

Depending on the kind of bacteria a patient to be a source of infection is infected with, a degree of reduction in infection risk (ultraviolet irradiation time in each room after the exit of the patient) is appropriately selected. The control is made in such a manner that ultraviolet irradiation is performed in each room for two minutes if it is desired to reduce the infection risk to one tenth, for four minutes if one hundredth, and for about eight minutes if one ten-thousandth, for example, and then (after the passage of such an interval) a patient is allowed to move to the next room.

Note that ultraviolet irradiation in the waiting room S1 is limited to time before accepting patients since the acceptance of patients cannot be restricted. However, since the air disinfected in the medical interview and examination room S2 and the treatment room S3 flows into the waiting room S1 in accordance with a pressure loss in each room, air contamination can be suppressed. Its effectiveness is significant since the infection risk caused by the previous patient can be reduced in the medical interview and examination room S2 and the treatment room S3 by the above-described turning-on and turning-off control program.

The simplified (for example, prefabricated or modular) medical facility in FIG. 8B may be a makeshift facility or a makeshift tent, or may be an ultraviolet irradiation unit capable of integrally moving or being set up with the ultraviolet irradiation device 100 of the present embodiment.

An ultraviolet irradiation system may be configured by combining the above-described ultraviolet irradiation device 100 with a management unit. The management unit in this case is a unit for managing the entry and exit of a person to and from the area S to be disinfected (for example, a unit that manages entry to and exit from the room). The drive control unit 113 of the ultraviolet irradiation device 100 performs the control of the ultraviolet irradiation device 100 in conjunction with the entry and exit management (management for entry to and exit from the room) made by the management unit.

Specifically, once an IC card for entering a room is read by a card reader provided outside of the room, for example, the door of the room becomes openable (the person can enter the room), and the ultraviolet irradiation device 100 is simultaneously turned off. Once an IC card for leaving the room is read by a card reader provided inside of the room, the door of the room is opened again (the person can leave the room), and then (after the door is closed following the exit, for example) the ultraviolet irradiation device 100 is turned on. In this case, although the ultraviolet irradiation device 100 may include no human detection sensor 115, the human detection sensor 115 may be provided for double safety management.

In each of the examples shown in FIGS. 8A and 8B, the initial ultraviolet irradiation time (the first time T1) is also determined depending on how much the initial number of bacteria should be reduced (where to place the limit of infection risk). For example, two minutes is selected if it is desired to reduce the infection risk to one tenth, four minutes if one hundredth, about eight minutes if one ten-thousandth, about 10 minutes if one hundred-thousandth, and so forth.

Note that the irradiation (turning-on) time (UV illuminance) and the turning-off time in the above-described turning-on and turning-off control program are given by way of example. Depending on the amount of estimated introduced (falling) bacteria and the kind of such bacteria, the size of the area S to be disinfected, the ventilation capacity of the area S to be disinfected, the room capacity, and the level of disinfection treatment according to the conventional method, setting (timing) capable of achieving efficient disinfection and preventing infection (suppressing bacterial growth for the irradiation in the second round (second cycle) and subsequent rounds) is appropriately selected.

In addition to the irradiation (turning-on) time (UV illuminance) and the turning-off time, setting for an irradiation direction and air conditioning (flow of the air) in the area S to be disinfected is appropriately selected so that efficient disinfection can be performed and infection can be prevented (bacterial growth can be suppressed).

If a member such as a safety cover or a protection film intervenes in an ultraviolet irradiation path, the transmittance of the member can be taken into consideration as appropriate. Specifically, to calculate a cumulative amount of light (μj/cm2) in the above-described (Expression 1), the following (Expression 2) that takes the transmittance of the irradiated material and a safety coefficient into consideration can be employed.

Cumulative amount of light (μj/cm2)=UV illuminance (μW/cm2)×cumulative irradiation time (sec)×transmittance (%) of material×safety coefficient (Expression 2)

The safety coefficient as used herein refers to a coefficient based on a degree of lamp consumption, a factor of safety, etc. The safety coefficient is a value calculated assuming that the illuminance of the lamp at the end of life is 70% of the initial lamp illuminance. The sanitization treatment by UV can inactivate pathogenic microorganisms by damaging their DNA and RNA, thereby reducing infection risk. The sanitization treatment by UV, on the other hand, may recover a function as a cell by different light energy. The cause of such recovery is believed to be the action of an enzyme present in a cell. This occurs by such a reaction that dimers, such as thymine, generated by UV irradiation having a wavelength of 253.7 nm are cleaved into the original bases by the action of near-ultraviolet light energy around 360 nm. That is, it is believed that the photoreactivation of bacteria occurs by the irradiation of light in a region having energy near 360 nm as a dominant wavelength.

It is believed that no such photoreactivation occurs in viruses having relatively simple cell structures. Bacteria such as Escherichia coli and microorganisms, however, have such enzymes. That is, in order to provide sufficient risk management while recognizing that some of these pathogenic microorganisms have the ability of photoreactivation, the principle of photoreactivation and its recovery rate are preferably taken into consideration. When photoreactivation is taken into consideration, for example, an energy amount necessary for disinfection can be calculated by doubling the energy amount shown in FIG. 4. Taking Escherichia coli as an example, a cumulative amount of light twice as much as a cumulative amount of light of 5,400 μJ/cm² to be required for disinfection can be used as an energy amount necessary for disinfection taking photoreactivation into consideration.

Although the above-described examples illustrate the cases where the area S to be disinfected is an operating room of a hospital, a waiting room, an examination room, or a treatment room, the area S to be disinfected may be an aseptic room (aseptic packaging room). The hospital may be an animal hospital, for example.

Alternatively, the area S to be disinfected may be a clean room in which precision instruments are manufactured, medical drugs are manufactured, or food processing (especially the processing of food or the like with no use of a preserving agent, or aseptic packaging process) is performed, for example.

The attachment position of the ultraviolet irradiation unit (low-pressure mercury lamp) 112 is not limited to the ceiling. The ultraviolet irradiation unit 112 may be installed on a wall surface, a floor surface, a post surface, a front surface of an illuminating lamp, or an inner surface or an outer surface of a translucent protection cover. The ultraviolet irradiation unit 112 is not limited to a type attached to the area S (the inside of a room) to be disinfected (stationary type). The ultraviolet irradiation unit 112 may be of a portable type (handy type).

Although the straight-tube low-pressure mercury lamp has been described as an example of the ultraviolet irradiation unit 112, the shape of the low-pressure mercury lamp is not limited to the illustrated example. For example, a bulb-type low-pressure mercury lamp may be employed.

With regard to the UV intensity of the ultraviolet irradiation unit (low-pressure mercury lamp) 112, no uniform standards have been set by The Illuminating Engineering Institute of Japan. In addition, a light receiving element may be deteriorated by UV. Thus, UV intensity may be controlled by performing calibration each time using a given instrument in order to obtain an accurate value constantly.

As a method for measuring the intensity of ultraviolet necessary for disinfection, a portable UV illuminance meter having a sensitivity peak in a range of 260 nm to 265 nm is commercially available, and the UV illuminance meter can be carried to a place where disinfection treatment is needed to measure if UV intensity having energy of 253.7 nm is being obtained. The actual bactericidal effect and necessary ultraviolet irradiation amount can be assessed by validation performed in conjunction with, for example, a microbial detection method (Japan Food Research Laboratories) according to which germs such as falling bacteria are collected and cultured on a petri dish and then the number of bacteria is measured.

The ultraviolet irradiation unit 112 of the present embodiment may be any UV light source as long as the UV light source can generate an output wavelength corresponding to energy in the short-wavelength UVC region of ultraviolet light, and has the ability to inactivate germs by directly destroying DNA of the germs. A light emitting diode (LED) UV lamp may be employed, for example.

Typical examples of light sources, other than mercury lamps, capable of outputting ultraviolet include UV-LEDs containing no mercury and capable of obtaining energy in the ultraviolet region. Among such UV-LEDs, a UV-LED light source having an emission line in a range from the UVC region to the UVB region, in particular, in a range from 260 nm to 285 nm, and capable of obtaining a single wavelength has characteristics such that its luminous efficiency is excellent and its illuminance is less likely to deteriorate, as well as a long life. The energy output of such a UV-LED light source corresponds to the energy output of UVC, which is the bactericidal wavelength region shown in FIG. 3. That is, high bactericidal effect can be obtained also in such a UV-LED light source. Thus, a UV-LED may be employed instead of the low-pressure mercury lamp 112 as an embodiment of the present invention.

### Illumination Device

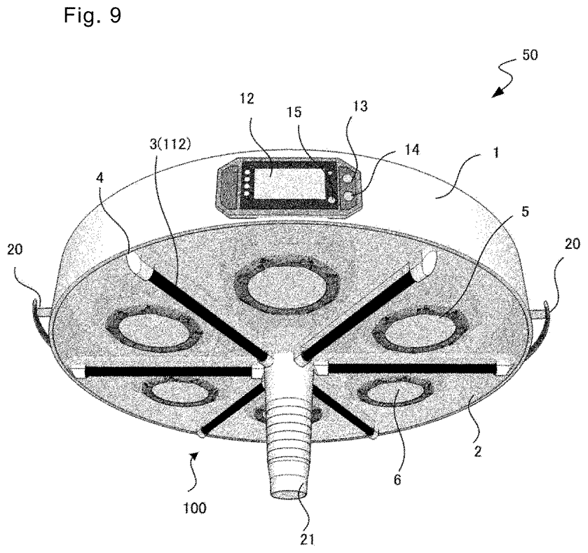

With reference to FIGS. 9 to 13, an illumination device 50 including the above-described ultraviolet irradiation device 100 and illumination light sources 6 will be described next taking the illumination device (shadowless lamp) 50 used in an operating room as an example.

The above-described embodiment has described a case in which the number of bacteria is controlled (growth is suppressed) uniformly over the area S (such as the inside of a room) to be disinfected. In a place such as an operating room, however, the number of bacteria may be suppressed more efficiently if the area S to be disinfected can also be narrowed down to a patient (the surgical field of the patient, in particular) so that ultraviolet can be intensively applied thereto.

In an operating room, a patient is placed on an operating table disposed in the center of the operating room, unnecessary portions of patient's clothes are taken off, and the patient is sanitized with a strong disinfectant, and then entirely covered with an aseptic drape. Thereafter, only a portion of the drape corresponding to a region to be operated is cut off, and a surgical instrument operated, for example, by a doctor or an operator's hand penetrates into the region to be operated. If bacteria are introduced into this region, there is a risk that the bacteria go into the body of the patient and the patient becomes infected with the bacteria. That is, the portion of the surgical field and a space above such a portion in this case become the area S to be disinfected that requires an aseptic state very strongly.

If a patient is infected with a kind of bacteria, the operating table and its surrounding area are more likely to be contaminated by the kind of bacteria. Also in this case, the patient and its surrounding area become an area (area S to be disinfected) especially desired to undergo intensive disinfection.

In order to perform disinfection treatment on such an area S to be disinfected by intensively applying ultraviolet thereto, the above-described ultraviolet irradiation device 100 is incorporated into the illumination device (shadowless lamp) 50 that is located directly above the operating table, capable of reliably delivering light to required areas, and designed to illuminate a surgical field without creating a shadowed area as much as possible.

That is, the illumination device 50 of the present embodiment functions as lighting (shadowless lamp) for surgery, and also irradiates the area S to be disinfected (surgical field) with an appropriate amount of ultraviolet as needed. In addition, during a period of time not involving surgery, the illumination device 50 functions as the ultraviolet irradiation device (disinfection device) 100 for irradiating the inside of the operating room including the operating table (in this case, the inside of the operating room also becomes the area S to be disinfected) with ultraviolet. Thus, the illumination device 50 can disinfect the surgical field of the patient while providing illumination thereto during surgery. After the surgery, the illumination device 50 can effectively disinfect viruses or germs attached to the surfaces of equipment items as a result of airborne droplets or contact as well as a variety of bacteria such as falling bacteria and airborne bacteria.

The illumination device 50 will be specifically described below. FIG. 9 is an external perspective view of the illumination device (shadowless lamp) 50 according to the present embodiment.

As shown in FIG. 9, the shadowless lamp 50 of the present embodiment includes: the illumination light sources (halogen lamps or white LEDs) 6; the ultraviolet irradiation device 100; a front clear cover 2; a main body unit (case) 1; an angle adjustment grip 21; side grips 20; and an operation panel 12, for example.

The main body unit 1 integrates the whole shadowless lamp 50. The side grips 20 are provided on both sides of the main body unit 1, and the angle adjustment grip 21 is provided so as to protrude from a central portion of the main body unit 1. The side grips 20 and the angle adjustment grip 21 are provided for the purpose of appropriately adjusting the position of the shadowless lamp 50 as desired during surgery so as to apply optimal illumination to an affected area.

The illumination light sources 6 are spaced out evenly over the front surface of the main body unit 1 and covered with the front clear cover 2. A condensing lens (illumination lens) 5 capable of irradiating an affected area with an optimal amount of light by a procedure and an adjustment dial (not shown) for the condensing lens 5 are provided over the surface thereof in an irradiation direction of light 22 from the illumination light source 6.

The front clear cover 2 for protecting the front surface of the shadowless lamp 50 prevents airborne droplets containing bacteria from being attached to the shadowless lamp 50. The front clear cover 2 employs a material such as quartz glass or a fluorocarbon resin material as a material not interrupting light as illumination and capable of withstanding ultraviolet.

The ultraviolet irradiation device 100 includes UV lamps (such as low-pressure mercury lamps, or UV-LEDs, for example) 3 as the ultraviolet irradiation units 112 capable of outputting light with energy in the wavelength range of the UVC region having high bactericidal effect. The configuration of the ultraviolet irradiation device 100 is the same as that described above in FIG. 1, etc. except that the UV lamps 3 are disposed on the front surface of the front clear cover 2 alternately with the illumination light sources 6.

A lighting change-over switch 13 for controlling the illumination light sources 6, and the operation panel 12 with which various operations such as adjusting the illuminance of shadowless light can be performed are provided on a side surface of the main body unit 1. The lighting change-over switch 13 is a switch for turning on and turning off the plurality of illumination light sources 6 individually so that a surgical field is irradiated uniformly, for example. The lighting change-over switch 13, however, may also have a function to switch between the lighting of the UV lamps 3 only and the lighting of the illumination light sources 6 only, for example.

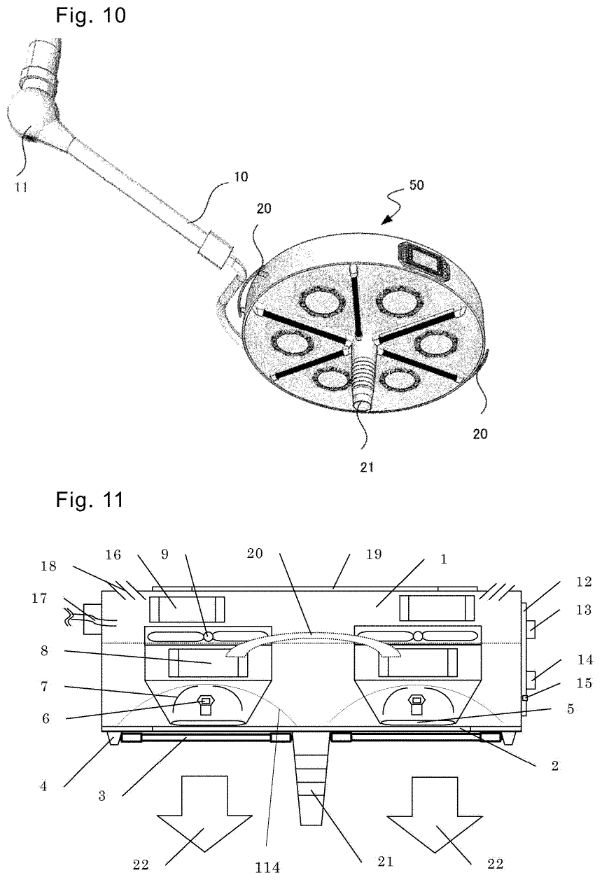

Referring to FIG. 10, the main body unit 1 is supported by an arm 10 and an arm articulation 11 serving as support members selected under strength design capable of supporting the weight of the main body unit 1. The main body unit 1 can be three-dimensionally moved not only from side to side or up and down but also diagonally. The position of the main body unit 1 can be changed freely by the side grips 20 as a worker (medical worker) who carries out surgery chooses. Also in the main body unit 1 itself, the irradiation angle of the shadowless lamp 50 can be changed as desired by the angle adjustment grip 21 in order to illuminate an operation site accurately during surgery, and fine adjustment of such an irradiation angle is possible too.

For the purpose of weight reduction, a resin material is mainly used as a material for forming surface cover portions of the main body unit 1, the arm 10, the side grips 20, and the angle adjustment grip 21, for example. Metal is used partially in supporting materials to keep body shapes and electric parts, for example. As mentioned above, a quartz glass material or a fluorocarbon resin is selected for the portion of the front clear cover 2.

Referring to FIGS. 9 and 11, the main body unit 1, the front clear cover 2, and a rear lid 19 each have a circular outer shape. In the main body unit 1, the illumination light sources (halogen lamps (white LEDs in some cases)) 6, the illumination lenses 5 for controlling focuses of illumination light of the illumination light sources 6, reflectors 7 for reflecting light from the illumination light sources 6, ballast power sources (electronic printed boards, lighting circuits for the illumination light sources 6) 8 for controlling the lighting of the illumination light sources 6, and cooling fans 9 for keeping temperature in the main body unit 1 constant in order to optimally turn on and drive the illumination light sources 6 and the ballast power sources 8 are each unitized and accommodated inside.

The front clear cover 2 and the rear lid 19 are each configured so as to be removable from the main body unit 1. Mounting parts into the main body unit 1 and maintenance of the parts can be easily performed from the front clear cover 2 side and the rear lid 19 side. The front clear cover 2 fits to the front surface of the main body unit 1. The rear lid 19 is assembled to the back surface of the main body unit 1 so that constitutes a surface cover portion (case member) integrally with the main body unit 1. Such a surface cover portion generally has a flat surface, and is configured with a material and a shape to which dust is less likely to stick and that are easily subjected to wipe cleaning.

Ballast power sources 16 of the ultraviolet irradiation device 100 are also accommodated in the main body unit 1. The ballast power source 16 in this case is an electronic printed board or a UV lamp lighting circuit, and is a part of the above-described drive control unit 113.

The ballast power sources 8 and 16 are accommodated in the main body unit 1 or in a box connected via the arm 10, and thus the circuits for efficiently turning on the illumination light sources 6 and the UV lamps 3 are installed integrally with the illumination device 50.

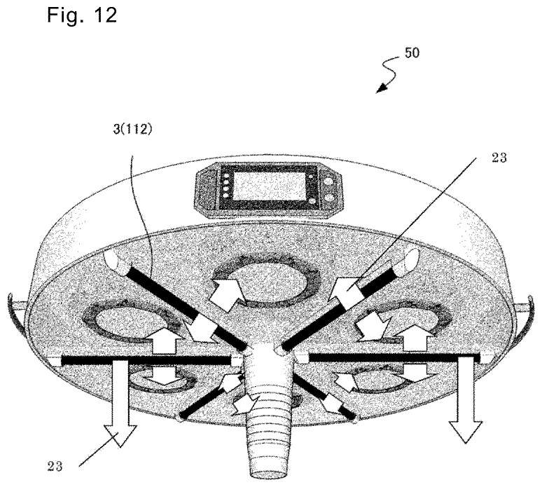

The UV lamps 3 are attached to the front clear cover 2. The UV lamps 3 are arranged on the front surface of the front clear cover 2 having a generally circular shape alternately with the illumination light sources 6 at equal intervals along the circumferential direction of the front clear cover 2 (see FIG. 9). Thus, the irradiation direction of the light 22 of the illumination light source 6 (see FIG. 11) and at least some of irradiation directions of ultraviolet 23 of the UV lamp 3 (see FIG. 12) are set to the same direction. If the condensing unit 114 is provided, the ultraviolet 23 of the UV lamp 3 is irradiated thoroughly in the same direction as the irradiation direction of the light 22 of the illumination light source 6 also by the condensing unit 114.

Specifically, the condensing unit (such as a reflector, for example) 114 is provided on the rear side of the UV lamp 3 in the main body unit 1 so as to be shape-changeable, for example. Thus, irradiation can be directed to all of the ceiling, walls, floor, and space of an operating room as well as equipment items present in the operating room. In addition, irradiation can be directed to the surgical field of a patient in a concentrated manner. Switching between these irradiation directions can be done by the movement of the condensing unit 114, for example.

Furthermore, irradiation of the ultraviolet 23 from the UV lamp 3 travels also along the front surface of the front clear cover 2 in the shadowless lamp 50 (see FIG. 12). Thus, the front surface of the front clear cover 2 can also be disinfected.

The irradiation direction of the light 22 of the illumination light source 6 and the irradiation direction of the ultraviolet 23 of the UV lamp 3 can be each changed as desired by the angle adjustment grip 21. Alternatively, the shadowless lamp 50 may be configured so that such irradiation directions can be each adjusted by an operation via the operation panel 12, for example.

Since the main body unit 1 is a housing closed in all directions as described above, the illumination light sources 6, the ballast power sources 8, the ballast power sources 16, etc., accommodated therein become heat sources. Thus, there is a risk that the accumulation of heat damages these parts and disturbs ambient temperature during surgery even in an air-conditioned room. In order to cool these parts forcibly, the cooling fan 9 is provided above the illumination light source 6 and the ballast power source 8 so as to discharge heat in the main body unit 1 constantly. The cooling fans 9 as many as the illumination light sources 6 are installed so as to correspond to the illumination light sources 6, and the inside of the main body unit 1 can be cooled by constant ventilation through louvers 18 provided at side edges of the rear surface of the main body unit 1.

The illumination light source 6 is provided on an insulator (a printed board in the case of an LED), and a circuit wire-connected from the insulator (printed board) through the lighting circuit such as the ballast power source 8 to a facility-side power supply port (not shown) provided outside of the main body unit 1 is formed. Wire connection between the electric parts is made so as to be detachable by a dedicated connector when appropriate, thereby making it easy to replace the parts when damaged and perform maintenance check. The UV lamp 3 and the ballast power source 16 are also wire-connected in a similar manner. For such wire connection, a detachable connector part is employed.

A wire 17 connected to the external facility-side power supply port (not shown) from each electric part in the main body unit 1 has a structure capable of being connected to the facility-side power supply port by making use of a hollow space inside the arm 10 and the arm articulation 11 for holding the main body unit 1 since the wire 17 would get in the way of a worker who carries out surgery if exposed to the outside.

To meet the need for reliably providing various states of light responding to all sorts of surgery, for example, the main body unit 1 of the present embodiment is configured (FIG. 12) as a ceiling-suspended type, and configured so as to be supported by the arm 10 and the arm articulation 11 having excellent movability and high accuracy, and to be adjustable for its position. The main body unit 1, however, is not limited thereto, and can be easily modified (applied) to a ceiling-buried type configuration or a configuration supported by a self-standing support member depending on the situation on the ground.

The shadowless lamp 50 of the present embodiment is lighting mainly used when surgery is carried out in a medical facility. The shadowless lamp 50 of the present embodiment is also medical special lighting capable of irradiating an affected area of a patient (i.e., an object to be irradiated) intensively with a plurality of light sources so as not to create shadows and capable of being adjusted to have optimal illuminance and irradiation angle so that treatment operations can be smoothly carried out by a doctor and a nurse who are practitioners.

The illumination light sources (halogen lamps or white LEDs) 6 of the shadowless lamp 50 are spaced out evenly over the front surface of the main body unit 1. The light distribution of the illumination light sources 6 is designed so that illumination can be efficiently provided to a patient having surgery.

Specifically, in order to ensure illuminance with which a doctor (operator) can observe an affected area best when carrying out surgery, light distribution for the light 22 from the shadowless lamp 50 is basically designed so that a straight-ahead direction can be illuminated by a light source unit including the high-intensity halogen lamp (white LED in some cases) 6, the reflector 7 for optimizing light distribution performance, and the illumination lens 5 for regulating light scatter. In addition to such basic light distribution design, a mode in which a patient is illuminated wholly, and a mode in which moderate spot light irradiation can also be provided by focusing on a specific target site of a patient in some instances, for example, can be switched therebetween. Such switching control is performed by the touch-sensitive operation panel 12 (or the change-over switch 13), for example. The operation panel 12 is provided, for example, on the side surface of the main body unit 1 so that a worker can smoothly make such switching even during a procedure.

The UV lamps 3 of the ultraviolet irradiation device 100 integrated in the shadowless lamp 50 of the present embodiment are straight-tube UV lamps 3 capable of efficiently outputting ultraviolet in the UVC region, which is bactericidal energy, for example. The UV lamp 3 is attached to the front surface of the main body unit 1 (at a position on the same level as the front clear cover 2) by a lamp holder 4. Note that the plurality of illumination light sources 6 and the plurality of UV lamps are disposed alternately in the circumferential direction of the front clear cover 2 having a generally circular shape.

FIG. 12 is a schematic view showing the layout of the UV lamps 3 and the irradiation directions of the light (ultraviolet) 23 irradiated from the UV lamps 3. The straight-tube UV lamp 3 is capable of light irradiation in all directions as with light of a typical fluorescent lamp. Thus, the bactericidal energy of the UV lamps 3 can be spread in directions along the front surface of the front clear cover 2 of the main body unit 1, over the working space for surgery, and in the same direction as the irradiation direction (FIG. 11) of the light 22 provided by the shadowless lamp 50 as illumination. This can effectively disinfect harmful falling bacteria attached mainly to the shadowless lamp 50 and an operating table as work instruments as well as the surfaces of their grab handles, and airborne bacteria in a short amount of time, thus achieving infection prevention.

Since the ultraviolet energy itself that is effective for disinfection is harmful to the human body, it is desirable to have a configuration that prevents the direct application of ultraviolet to a doctor and a nurse carrying out surgery as well as a region of a patient excluding a surgical field, in particular.

In view of the foregoing, while ensuring, as the shadowless lamp 50, brightness under which normal surgery can be carried out with no difficulty, turning-on and turning-off of the shadowless lamp 50 (the illumination light sources 6) and turning-on and turning-off of the UV lamps 6 are made switchable so that the UV lamps 3 attached to the front surface thereof (the surface of the front clear cover 2) can be turned on and off independently of the shadowless lamp 50. Such switching between the turning-on and turning-off of the shadowless lamp 50 and the turning-on and turning-off of the UV lamps 3 can be manually done by the change-over switch 13, for example. Moreover, a timer function by which irradiation time of the UV lamps 3 can be automatically set (controlled) may be provided. Furthermore, the UV lamps 3 have a control program for their turning-on time and turning-off time in the drive control unit 113 of the ultraviolet irradiation device 100. The control program for the turning-on time and turning-off time of the UV lamps 3 is similar to that described as a configuration of the above-described ultraviolet irradiation device 100. That is, a method for controlling the turning-on time and turning-off time of the UV lamps 3 is set on the basis of the above-described control method in the ultraviolet irradiation device 100. Setting for the turning-on time and turning-off time of the UV lamps 3 as well as timer setting for irradiation time can also be made or changed as desired (manually) by an operation via the operation panel 12 or the change-over switch 13, for example.

Ultraviolet irradiation (turning on) by the UV lamps 3 is performed, for example, by a control program (or manually) on the inside of an operating room, an operating table, other equipment items, and a space during a period of time conducting no surgery (a period of time in which the area S to be disinfected is unoperated). However, even during the operation of the area S to be disinfected, it is also possible to apply intensive irradiation to the surgical field of a patient having surgery, for example, only for a very short amount of time (for an amount of time short enough to have no adverse effects on the human body), such as before suturing. Consequently, falling bacteria in the surgical site of the patient and airborne bacteria around the surgical site can be disinfected. Specifically, operation as with the turning-on and turning-off control program described with reference to FIG. 8A, for example, is possible.

First of all, the number of bacteria in the operation area can be reduced before opening the chest (making an incision) of a patient by using the ultraviolet irradiation device 100 of the present embodiment as described above. Moreover, increase in the number of bacteria can be suppressed during the surgery by allowing workers to be evacuated only for a short amount of time in order to perform disinfection. Thus, the area S to be disinfected can be disinfected with the least possible interruption of the surgery (increasing the operating rate of the operating room). Moreover, after the completion of the surgery, the surgical field can be irradiated with ultraviolet for a short amount of time before the chest is closed (the incision is closed). By doing so, the chest can be closed with the existing probability of bacteria being lowered, and thus the postoperative infection probability can be lowered.

Although it is possible to take sufficient time for disinfection treatment during a period of time conducting no surgery, entry of a worker to an operating room needs to be managed in this case. Thus, monitoring as to whether a person is present in the operating room, particularly near (directly under) the shadowless lamp 50, is performed by a human detection sensor 15 (115) provided in the ultraviolet irradiation device 100. The UV lamps 3 may be configured to be turned off automatically when the presence of a person is detected while the UV lamps 3 are on.

In addition to this, an emergency stop button 14 for enabling emergency stop when the shadowless lamp 50 (the illumination light sources 6) is mistakenly switched to the UV lamps 3 under the presence of a worker near the shadowless lamp 50 may be provided near the operation panel 12, for example. This can avoid adverse effects on the human body due to unintentional ultraviolet irradiation, and thus the disinfection treatment can be done safely.

As already mentioned, a resin material is mainly used for members constituting the main body unit 1 for the purpose of weight reduction. There is, however, a concern that the front clear cover 2, etc. are deteriorated by ultraviolet energy irradiated by the UV lamps 3 incorporated into the main body unit 1. For this reason, a part that constitutes the portion irradiated with ultraviolet may comprise a material such as soda glass that transmits no ultraviolet, aluminum whose surface having high durability against ultraviolet has been subjected to alumite treatment, and stainless steel.

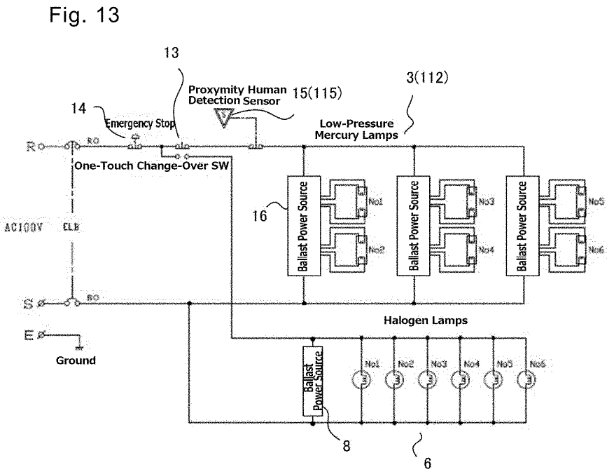

FIG. 13 is a circuit diagram showing an example of turning-on control of the shadowless lamp 50 (the illumination light sources 6) and the UV lamps 3 according to an embodiment of the present invention. Using a commercial AC 100V power source, which is easy to obtain at hospitals as a source of power, as an operating power source, a circuit for supplying power to the ballast power source 8 (printed board) necessary for turning on the illumination light sources 6 and a circuit (included in the drive control unit 113) for supplying power to the ballast power sources 16 (printed boards) necessary for turning on the UV lamps 3 are connected in parallel.

The numbers of the illumination light sources 6 and the UV lamps 3, as well as the numbers of the power sources necessary to turn on these light sources are appropriately selected in consideration of the necessary illumination space, the area to be disinfected (the size of the area S to be disinfected), the ventilation capacity, the room capacity, etc. That is, the numbers of the illumination light sources 6 and the UV lamps 3 are not limited to those shown in FIGS. 9 and 13 (six lamps each in both cases). These numbers may be increased or decreased.

Furthermore, the UV lamps 3 and the illumination light sources 6 may be configured so that any one of them can be individually selected and its turning-on and turning-off operations can be controlled. Such switching is performed via the change-over switch 13, for example. Thus, illumination or ultraviolet can be applied to a necessary region thoroughly without creating shadows by the irradiation by turning on and off the illumination light sources 6 and the UV lamps 3 as a group or individually one by one, for example.

Furthermore, a mode selection function by which at least any one of the UV lamps 3 and the illumination light sources 6 can be set to an optimal irradiation amount, and a power-saving mode selection function by which measures to restrain excessive irradiation of the UV lamps 3 during disinfection treatment, such as partially turning on (turning off) the UV lamps, can be performed as desired may be provided so that a worker can select such functions as desired. The control and selection of these functions are performed by operations via the operation panel 12, for example. Thus, such a device can be used for a wide variety of applications as the illumination device 50, capable of serving also as the ultraviolet irradiation device 100, more suited for the situation on the ground.

Since the bactericidal effect by ultraviolet is determined by a cumulative irradiation amount (mJ/cm²) as mentioned above, prescribed disinfection can be achieved by prolonging irradiation time (sec) in the case of low UV illuminance (mw/cm²) due to the power-saving mode. Therefore, necessary bactericidal effect can be reliably obtained by calculating irradiation time corresponding to an irradiation amount (%) obtained on the assumption that the partially turned off UV lamps 3 were being lighted, and turning on the remaining UV lamps longer by an amount corresponding to that irradiation time as setting of the timer for turning on the UV lamps 3.

A humidifier, a heater, a cooling unit, and the like may be provided in the main body unit 1, and humidification, heating, cooling, and the like may be performed on the surgical site of a patient as needed.

As described above, the shadowless lamp 50 of the present embodiment includes the UV lamps 3 capable of efficiently emitting ultraviolet energy in the UVC region, and thereby irradiates the surface of the illumination device and its surrounding space thoroughly with ultraviolet light irradiated from the UV lamps and having ability to kill bacteria. Thus, the shadowless lamp 50 of the present embodiment can perform disinfection treatment over a wide area by thoroughly irradiating a space in an operating room, an operating table, and instruments therearound with ultraviolet at the same time not only during surgery but also during a period of time not involving surgery.

Thus, the illumination device 50 capable of: eliminating burdens related to the disinfection treatment according to the conventional methods such as cleaning with an antiseptic solution (for example, spraying and wiping out a medical solution); automatically disinfecting the surgical environment; and maintaining a clean state can be provided.

That is, the illumination device 50 can disinfect a target instrument and its surrounding space in an area required to control the number of bacteria efficiently and safely without lowering the operating rate of the equipment, and can maintain such an environment.

For the use during surgery, the shadowless lamp 50 is generally installed in an operating room (over an operating table). The shadowless lamp 50 of the present embodiment, however, is expected to be widely used as an illumination device not only in an ordinary operating room but also in an intensive care unit, or in an experimental operation or in an animal hospital, in some cases. The function to disinfect a space in an operating room and the surfaces of equipment items such as an operating table, which is provided by the ultraviolet irradiation device 100 of the present embodiment, in particular, can protect even seriously ill patients, the aged, and children with weak physical strength against infection, and can eliminate the source of infection itself without bothering doctors or nurses, who are workers on the ground, thereby improving the hygienic level of the work environment.

The above-described illumination device 50 of the present embodiment can be applied to illumination devices used in treatment rooms, aseptic packaging rooms, or animal hospitals, or illumination devices used, for example, in clean rooms in which precision instruments are manufactured, medical drugs are manufactured, or food processing (especially the processing of food or the like with no use of a preserving agent, or aseptic packaging process) is performed, for example, without being limited to shadowless lamps.

Note that the present invention is not limited to the above-described embodiments, and various modifications can be made thereto without departing from the scope of the present invention.

### INDUSTRIAL APPLICABILITY

The present invention can be utilized for a disinfection device in an environment required to control the number of bacteria, such as an operating room or a clean room.

1. An ultraviolet irradiation device configured to perform disinfection by irradiating an area to be disinfected with ultraviolet, comprising:
an ultraviolet irradiation unit capable of outputting ultraviolet having a predetermined dominant wavelength; and
a drive control unit, wherein
the drive control unit performs time control of ultraviolet irradiation and non-irradiation by the ultraviolet irradiation unit on a basis of time necessary to disinfect the area to be disinfected before or during operation and bacterial growth time after the disinfection.

2. The ultraviolet irradiation device according to claim 1, wherein
the drive control unit controls the ultraviolet irradiation unit so that ultraviolet is applied in a first disinfection treatment of the area to be disinfected during first time and after that, a non-irradiation state is maintained during second time,
the first time is an amount of time during which the first disinfection treatment can be performed,
the second time is an amount of time during which growth of a predetermined kind of bacterial after passage of the first time can be suppressed, and
the second time is longer than the first time.

3. The ultraviolet irradiation device according to claim 2, wherein
the drive control unit controls the ultraviolet irradiation unit so that ultraviolet is applied again after passage of the second time and non-irradiation is performed again at least once,
an amount of time during which ultraviolet is applied again is third time,
an amount of time during which non-irradiation is performed again is fourth time,
the third time is an amount of time during which increased bacteria after passage of the second time can be disinfected,
the fourth time is an amount of time during which bacterial growth after the passage of the third time can be suppressed, and
the fourth time is longer than the third time.

4. The ultraviolet irradiation device according to claim 3, wherein the third time is shorter than the first time.

5. The ultraviolet irradiation device according to claim 1, comprising a detection unit configured to detect presence or absence of a person at least in an ultraviolet irradiation region in the area to be disinfected, and wherein
when the detection unit detects the presence of a person, the drive control unit turns the ultraviolet irradiation unit into a non-irradiation state.

6. The ultraviolet irradiation device according to claim 1, wherein the ultraviolet irradiation unit outputs ultraviolet having a wavelength in a UVC region.

7. The ultraviolet irradiation device according to claim 1, comprising an inhibition unit configured to inhibit generation of ozone due to irradiation of air with ultraviolet.

8. The ultraviolet irradiation device according to claim 1, comprising a plurality of the ultraviolet irradiation units.

9. The ultraviolet irradiation device according to claim 1, wherein the area to be disinfected is a working space into which a worker can enter or from which the worker can leave and which is expected to keep a predetermined level of cleanliness.

10. An ultraviolet irradiation method for performing disinfection by irradiating an area to be disinfected with ultraviolet, comprising:
performing time control of irradiation and non-irradiation with ultraviolet having a predetermined dominant wavelength on a basis of time necessary to disinfect the area to be disinfected during operation and bacterial growth time after the disinfection.

11. The ultraviolet irradiation method according to claim 10, comprising the steps of:
applying ultraviolet in a first disinfection treatment of the area to be disinfected during a first time; and
maintaining a non-irradiation state of ultraviolet during a second time after passage of the first time, and wherein
the first time is an amount of time during which the first disinfection treatment can be performed,
the second time is an amount of time during which growth of a predetermined kind of bacterial after passage of the first time can be suppressed, and
the second time is longer than the first time.

12. The ultraviolet irradiation method according to claim 11, comprising the steps of:
applying ultraviolet again during a third time after passage of the second time; and
maintaining non-irradiation again during a fourth time after passage of the third time, and wherein
the third time is an amount of time during which increased bacteria after passage of the second time can be disinfected,
the fourth time is an amount of time during which bacterial growth after the passage of the third time can be suppressed, and
the fourth time is longer than the third time.

13. The ultraviolet irradiation method according to claim 12, wherein the third time is shorter than the first time.

14. The ultraviolet irradiation method according to claim 10, comprising detecting presence or absence of a person at least in an ultraviolet irradiation region in the area to be disinfected; and when the presence of a person is detected, turning the ultraviolet into a non-irradiation state.

15. The ultraviolet irradiation method according to claim 10, wherein the ultraviolet has a wavelength in a UVC region.

16. The ultraviolet irradiation method according to claim 10, wherein ultraviolet of wavelengths that generate ozone out of the ultraviolet is removed for irradiation of air.

17. An illumination device comprising:
the ultraviolet irradiation device according to claim 1; and
an illumination light source.

18. The illumination device according to claim 17, wherein an irradiation direction of light of the illumination light source and at least some of irradiation directions of ultraviolet of the ultraviolet irradiation device are set to same directions.

19. The illumination device according to claim 18, comprising an adjustment unit capable of changing the irradiation direction of light and the irradiation directions of ultraviolet.

20. The illumination device according to claim 17, wherein the illumination light source is any of a halogen lamp and an LED and has a function of a shadowless lamp.

21. An ultraviolet illumination system comprising:
the ultraviolet irradiation device according to claim 1; and
a management unit configured to manage entry and exit of a person to and from the area to be disinfected, wherein
the drive control unit performs control of the ultraviolet irradiation device in conjunction with entry and exit management made by the management unit.
